SEQUENCE LISTING (6 sequences)

SEQ ID NO: 1 - DNA, 1928 bp, Homo sapiens
  CDS (92)..(1543)
ggcacgagctcttccgcacatgtgtctgctgtaaacacatgtctgtagttttttaagctcctgtctgaagtaaggcgacccttctgaataaatggtgtggatcgcggacgaggcggtggcgcgcgatgtgcagcggttgctagtgcagttccaggatgagggcgggcagctgctgggttccccgttcgacgtgcccgtggacatcaccccggacaggctgcagctcgtttgcaacgcgctactggcccaggaggatcccctgtcactggctttctttgtccacgatgctgagatcgtttcctcactcgggaagacgttggagtcccaggcagtggagacagagaaggtcctagacatcattttacccacacaagctgtgttcaaagttcgtgctgtaacacgatgtaccagctcattggagggacacaccgaggctgttatttcagtagccttcagcccaactggaaagtatttggcaagtggttctggggacactacagtccgcttttgggatctcagcacagaaactccacattttacatctaaagggcatacacactgggttctcagtattgcttggtctccagatggcaaaaaacttgcctcaggatgtaaaaatagtcagatcttcatttgggacccaagcacagggaagcagattggcaaaccattaacagggcactcaaagtggattacatggctgtgttgggaacctctccacctgaacccagagagccgatacctagccagtgcctccagcggccgcgtcgaccggatctgggacacaactgcaggccgctgtgagcgcatcctcaccgggcacacccagtcggtcacctgtctccggtggggaggggacgggcttctctactctgcctcccaggaccgcaccatcaaagtctggagagctcatgacggtgtgctgtgccggactctgcaaggccacggccactgggtgaacaccatggccctcagcactgactatgccctgcgcactggggcctttgaacctgctgaggcctcagttaatccccaagacctccaaggatccttgcaggagttgaaggagagggctctgagccgatacaacctcgtgcggggccagggtccagagaggctggtgtctggctccgacgacttcaccttattcctgtggtccccagcagaggacaaaaagcctctcactcggatgacaggacaccaagctctcatcaaccaggtgctcttctctcctgactcccgcatcgtggctagtgcctcctttgacaagtccatcaagctgtgggatggcaggacgggcaagtacctggcttccctacgcggccacgtggctgccgtgtaccagattgcgtggtcagctgacagtcggctcctggtcagcggcagcagtgacagcacactgaaggtgtgggatgtgaaggcccagaagctggccatggacctgcccggccacgcggatgaggtatatgctgttgactggagtccagatggccagagagtggcaagtggtgggaaggacaaatgcctccggatatggaggagatgagacggcccgaagttctctctgacccccacctcgactcggcctctgccagctgccttccctgccagagaacaaaggctgagatggcagtgcacacaccctccccaccagtggggacctgagaatgcgtgtggcctgctgtcctcgatagaccggaatggggttttcccacagatccccgcctgtggcacaccccagagccagaaatcgaaggtcacaggaagttgtcactgaacttggcccgtgtctgctactctgtaccttgctggtacagacaggggtggtgggcagccaggctctatgagtgggcccctagtgtcagctctgtacagggtcagatcccaggttctatgaccaaataagtaacttaaaaaaaaaaaaaaaa

SEQ ID NO: 2 - protein, 484 aa, Homo sapiens
MVWIADEAVARDVQRLLVQFQDEGGQLLGSPFDVPVDITPDRLQLVCNALLAQEDPLSLAFFVHDAEIVSSLGKTLESQAVETEKVLDIILPTQAVFKVRAVTRCTSSLEGHTEAVISVAFSPTGKYLASGSGDTTVRFWDLSTETPHFTSKGHTHWVLSIAWSPDGKKLASGCKNSQIFIWDPSTGKQIGKPLTGHSKWITWLCWEPLHLNPESRYLASASSGRVDRIWDTTAGRCERILTGHTQSVTCLRWGGDGLLYSASQDRTIKVWRAHDGVLCRTLQGHGHWVNTMALSTDYALRTGAFEPAEASVNPQDLQGSLQELKERALSRYNLVRGQGPERLVSGSDDFTLFLWSPAEDKKPLTRMTGHQALINQVLFSPDSRIVASASFDKSIKLWDGRTGKYLASLRGHVAAVYQIAWSADSRLLVSGSSDSTLKVWDVKAQKLAMDLPGHADEVYAVDWSPDGQRVASGGKDKCLRIWRR

SEQ ID NO: 3 - DNA, 1170 bp, Homo sapiens
  CDS (3)..(785)
gcggccgcgtcgaccggatctgggacacaactgcaggccgctgtgagcgcatcctcaccgggcacacccagtcggtcacctgtctccggtggggaggggacgggcttctctactctgcctcccaggaccgcaccatcaaagtctggagagctcatgacggtgtgctgtgccggactctgcaaggccacggccactgggtgaacaccatggccctcagcactgactatgccctgcgcactggggcctttgaacctgctgaggcctcagttaatccccaagacctccaaggatccttgcaggagttgaaggagagggctctgagccgatacaacctcgtgcggggccagggtccagagaggctggtgtctggctccgacgacttcaccttattcctgtggtccccagcagaggacaaaaagcctctcactcggatgacaggacaccaagctctcatcaaccaggtgctcttctctcctgactcccgcatcgtggctagtgcctcctttgacaagtccatcaagctgtgggatggcaggacgggcaagtacctggcttccctacgcggccacgtggctgccgtgtaccagattgcgtggtcagctgacagtcggctcctggtcagcggcagcagtgacagcacactgaaggtgtgggatgtgaaggcccagaagctggccatggacctgcccggccacgcggatgaggtatatgctgttgactggagtccagatggccagagagtggcaagtggtgggaaggacaaatgcctccggatatggaggagatgagacggcccgaagttctctctgacccccacctcgactcggcctctgccagctgccttccctgccagagaacaaaggctgagatggcagtgcacacaccctccccaccagtggggacctgagaatgcgtgtggcctgctgtcctcgatagaccggaatggggttttcccacagatccccgcctgtggcacaccccagagccagaaatcgaaggtcacaggaagttgtcactgaacttggcccgtgtctgctactctgtaccttgctggtacagacaggggtggtgggcagccaggctctatgagtgggcccctagtgtcagctctgtacagggtcagatcccaggttctatgaccaaataagtaacttaaaaaaaaaaaaaaaa

SEQ ID NO: 4 - protein, 261 aa, Homo sapiens
GRVDRIWDTTAGRCERILTGHTQSVTCLRWGGDGLLYSASQDRTIKVWRAHDGVLCRTLQGHGHWVNTMALSTDYALRTGAFEPAEASVNPQDLQGSLQELKERALSRYNLVRGQGPERLVSGSDDFTLFLWSPAEDKKPLTRMTGHQALINQVLFSPDSRIVASASFDKSIKLWDGRTGKYLASLRGHVAAVYQIAWSADSRLLVSGSSDSTLKVWDVKAQKLAMDLPGHADEVYAVDWSPDGQRVASGGKDKCLRIWRR

SEQ ID NO: 5 - DNA, 1859 bp, Homo sapiens
  miscellaneous feature 1154; 379: ATCG; variable amino acid
cgcccgggcaggtcgcaggatggcggcagcagtggcggacgaggcggtggcgcgcgatgtgcagcggttgctagtgcagttccaggatgagggcgggcagctgctgggttccccgttcgacgtgcccgtggacatcaccccggacaggctgcagctcgtgtgcaacgcgctactggcccaggaggatcccctgccactggctttctttgtccacgatgctgagatcgtctcctcactggggaagacgttggagtcccaggcagtggagacagagaaggtcctagacatcatctaccagccacaggctatcttcagagtccgggctgtgactcgctgcaccagctccttggagggtcacagtgaggcagtcatttctgtggccttcagccctacgggaaagtacctggccagtggctctggagacaccaccgtgcgcttctgggatctcagcacagagacaccacatttcacatgcaagggacacagacactgggtccttagtatatcctggtctccagatggcaagaagctggcctcaggctgcaagaatggccagattctcctctgggacccaagcacagggaagcaggtgggcaggaccctcgctggccacagcaagtggatcacaggcctgagctgggagcccctccatgcgaaccctgagtgccgctatgtggccagcagctccaaggatggcagtgtgcggatctgggacacaactgcaggccgctgtgagcgcatcctcaccgggcacacccagtcggtcacctgtctccggtggggaggggacgggcttctctactctgcctcccaggaccgcaccatcaaagtctggagagctcatgacggtgtgctgtgccggactctgcaaggccacggccactgggtgaacaccatggccctcagcactgactatgccctgcgcactggggcctttgaacctgctgaggcctcagttaatccccaagacctccaaggatccttgcaggagttgaaggagagggctctgagccgatacaacctcgtgcggggccagggtccagagaggctggtgtctggctccgacgacttcaccttattcctgtggtccccagcagaggacaaaaagcctctcactcggatgacaggacaccaagctctcatcaaccaggtgntcttctctcctgactcccgcatcgtggctagtgcctcctttgacaagtccatcaagctgtgggatggcaggacgggcaagtacctggcttccctacgcggccacgtggctgccgtgtaccagattgcgtggtcagctgacagtcggctcctggtcagcggcagcagtgacagcacactgaaggtgtgggatgtgaaggcccagaagctggccatggacctgcccggccacgcggatgaggtatatgctgttgactggagtccagatggccagagagtggcaagtggtgggaaggacaaatgcctccggatatggaggagatgagacggcccgaagttctctctgacccccacctcgactcggcctctgccagctgccttccctgccagagaacaaaggctgagatggcagtgcacacaccctccccaccagtggggacctgagaatgcgtgtggcctgctgtcctcgatagaccggaatggggttttcccacagatccccgcctgtggcacaccccagagccagaaatcgtaggtcacaggaagttgtcactgaacttggcccgtgtctgctactctgtaccttgctggtacagacaggggtggtgggcagccaggctctatgagtgggcccctagtgtcagctctgtacagggtcagatcccaggttctatgaccaaataagtaacttaaaaaaaaaaaaaaaa

SEQ ID NO: 6 - protein, 485 aa, Homo sapiens
  miscellaneous feature 379: variable amino acid
MAAAVADEAVARDVQRLLVQFQDEGGQLLGSPFDVPVDITPDRLQLVCNALLAQEDPLPLAFFVHDAEIVSSLGKTLESQAVETEKVLDIIYQPQAIFRVRAVTRCTSSLEGHSEAVISVAFSPTGKYLASGSGDTTVRFWDLSTETPHFTCKGHRHWVLSISWSPDGKKLASGCKNGQILLWDPSTGKQVGRTLAGHSKWITGLSWEPLHANPECRYVASSSKDGSVRIWDTTAGRCERILTGHTQSVTCLRWGGDGLLYSASQDRTIKVWRAHDGVLCRTLQGHGHWVNTMALSTDYALRTGAFEPAEASVNPQDLQGSLQELKERALSRYNLVRGQGPERLVSGSDDFTLFLWSPAEDKKPLTRMTGHQALINQVXFSPDSRIVASASFDKSIKLWDGRTGKYLASLRGHVAAVYQIAWSADSRLLVSGSSDSTLKVWDVKAQKLAMDLPGHADEVYAVDWSPDGQRVASGGKDKCLRIWRR

(19) United States
(12) Patent Application Publication
(10) Pub. No.: US 2003/0027261 A1
(43) Pub. Date: Feb. 6, 2003

(54) Novel genes TZap7/A, TZap7/B and TZap7 involved in T cell activation and uses thereof

(76) Inventor: Nalan Utku, Berlin (DE)

Correspondence Address: FISH & NEAVE, 1251 AVENUE OF THE AMERICAS, 50TH FLOOR, NEW YORK, NY, 10020-1105, US

(21) Appl. No.: 10/132,744

(22) Filed: Apr. 24, 2002

Related U.S. Application Data
(63) Continuation of application No. PCT/EP00/10670, filed on Oct. 30, 2000.
(60) Provisional application No. 60/162,675, filed on Nov. 1, 1999.
(60) Provisional application No. 60/185,016, filed on Feb. 25, 2000.

(51) Int. Cl. C07H 21/02; C07H 21/04; C12P 21/06

(52) U.S. Cl. 435/69.1; 536/23.1

(57) ABSTRACT

Described are generally immune response cDNA TZap7/A, TZap7/B and TZap7 polynucleotides encoding novel proteins involved in modulation of immune responses and antibodies recognizing said proteins. Furthermore, vectors comprising the aforementioned polynucleotides and host cells transformed therewith as well as their use in the production of the above-defined proteins are described. Additionally, pharmaceutical and diagnostic compositions are provided comprising any one of the afore-described polynucleotides, vectors, proteins, or antibodies. Furthermore, methods and uses for modulating immune responses through the novel TZap protein as well as pharmaceutical compositions comprising agents which act on the TZap protein or its ligand are described. Also, the use of said polynucleotides, vectors, proteins or antibodies for the preparation of diagnostic and pharmaceutical compositions for use in organ transplantation, for the treatment of autoimmune, allergic or infectious diseases, or for treatment of tumors is provided.

### FIELD OF THE INVENTION

The present invention pertains generally to TZap kinase 7 cDNAs TZap7/A, TZap7/B and TZap7 encoding novel immune response modulating proteins as well as peptides and polypeptides derived therefrom and antibodies recognizing said (poly)peptides. In a first aspect, the present invention relates to TZap7/A, TZap7/B and TZap7 cDNAs and their encoded proteins. In a further aspect, the present invention relates to polynucleotides derived from said TZap7/A, TZap7/B and TZap7 cDNAs encoding a peptide or polypeptide being capable of modulating immune responses. Furthermore, the present invention relates to vectors comprising such polynucleotides and host cells transformed therewith as well as their use in the production of the above-defined peptides or polypeptides. In addition, the present invention relates to the (poly)peptide encoded by said polynucleotides or obtainable by the method of the invention. In another important aspect the present invention relates to antibodies against said TZap7/A, TZap7/B and TZap7 proteins. Furthermore, the present invention relates to the use of antisense polynucleotides and vectors containing the same for the suppression of growth of leukocytes, in particular lymphocytes and monocytes. The present invention additionally relates to pharmaceutical and diagnostic compositions comprising the aforementioned polynucleotides, vectors, proteins, or antibodies.

Furthermore, the present invention relates to methods and uses for modulating immune responses through the novel TZap7/A, TZap7/B and TZap7 proteins as well as to pharmaceutical compositions comprising agents which act on the TZap7/A, TZap7/B and TZap7 proteins or their ligand. Also, the invention relates to the use of the before-described polynucleotide, vector, protein or antibody for the preparation of pharmaceutical compositions for use in organ transplantation, for the treatment of autoimmune, allergic or infectious diseases, or for treatment of tumors. Furthermore, the present invention relates to methods for modulating (antigen-specific) T, B, NK cell or monocyte (un)responsiveness.

Several documents are cited throughout the text of this specification. Each of the documents cited herein (including any manufacturer's specifications, instructions, etc.) are hereby incorporated herein by reference; however, there is no admission that any document cited is indeed prior art as to the present invention.

### BACKGROUND OF THE INVENTION

T cell activation is accompanied with sequential changes in the expression of various genes over several days and involves multiple signaling pathways [1]. Stimulation of T cells is initiated by the interaction of antigen-specific T cell receptors (TCR) with MHC bound antigenic peptides presented on the surface of antigen presenting cells (APC), but full proliferative T cell response requires additional costimulatory signals which are provided by the interaction of proteins expressed on the surface of T cells and APC [2,3,4,5]. In addition, a number of cytokines as well as other proteins are known to augment T cell activation, although many of them appear not to be essential for the basic proliferative T cell response [3,6]. Moreover, a growing body of evidence indicates that the microtubule cytoskeleton of lymphocytes plays a major role in T cell activation. Stimulation of T cells was demonstrated to result in molecular rearrangement in the actin cytoskeleton leading to re-localization and concentration of signaling molecules in restricted areas of the cell membrane close to the bound APC [7,8,9].

Although considerable information on T cell activation has been gathered in recent years, the complex molecular mechanisms of stimulation and signaling pathways are not completely understood. Since T cell activation provides the central event in various types of inflammation as well as in autoimmune disease and graft rejection, knowledge about the distinct steps and molecules involved in the stimulation process is of considerable biomedical importance, as they might provide targets for therapeutic modulation of the immune response. Therapeutic prevention of T cell activation in organ transplantation and autoimmune diseases presently relies on panimmunosuppressive drugs interfering with downstream intracellullar events. Specific modulation of the T cell response remains a longstanding goal in immunological research.

### SUMMARY OF THE INVENTION

The present invention relates to polynucleotides encoding a novel immune response modulating protein. Furthermore, the present invention relates to peptides and polypeptides derived therefrom as well as to antibodies. More particularly, the present invention relates to applications in the medical field that directly arise from the polynucleotides, protein, peptides, (poly)peptides and antibodies of the invention. Additionally, the present invention relates to a novel method for testing modulators of the immune response. The pharmaceutical compositions, methods and uses of the invention are useful therapeutically in situations where it is desirable to modulate (antigen-specific) immune responses, e.g., to induce and maintain (antigen-specific) T cell or B-cell unresponsiveness or restore (antigen-specific) B or T cell responsiveness. For example, it may be necessary to induce or maintain T cell unresponsiveness in a subject who has received an organ or bone marrow transplant to prevent graft rejection by inhibiting stimulation through the TZap7/A, TZap7/B and/or TZap7 protein. In addition, T cell unresponsiveness can be maintained by blocking TZap7/A, TZap7/B and/or TZap7 stimulation in a subject who has an autoimmune disease to alleviate symptoms of the autoimmune disease. In these cases, a TZap7/A, TZap7/B and/or TZap7 inhibitory agent is administered to the subject in an amount and over a period of time sufficient to maintain T cell unresponsiveness. Alternatively, T cell unresponsiveness can be reversed in a subject bearing a tumor to stimulate a tumor specific NK and T cell- response or in a subject receiving a vaccine to enhance the efficacy of the vaccine. For example, a cell (e.g., a tumor cell) can be modified to express a TZap7/A, TZap7/B and/or TZap7 ligand or a TZap7/A, TZap7/B and/or TZap7 stimulatory agent can be administered to the subject bearing a tumor or who has had a tumor surgically removed to prevent recurrence of the tumor.

### BRIEF DESCRIPTION OF DRAWINGS

This disclosure may best be understood in conjunction with the accompanying drawings, incorporated herein by references, which show:

FIG. 1: Human TZap B (Zap-consensus) amino acid sequence shares a high degree of identity with notchless protein in two different species (Xenopus and Drosophila) as shown in boxes.

FIG. 2: Human TZap A (Tzap7/A) amino acid sequence shows high degree of identity with notchless proteins in Xenopus and Drosophila.

FIG. 3: The amino acid sequence comparison between human TZap7 amino acid sequence and betatransducin proteins exhibits a number of motif-homology stretches to betatransducin protein within the 3′ region (boxes).

FIG. 4: Human TZap7 amino acid sequence shows high degree of identity with notchless proteins in Xenopus and Drosphila.

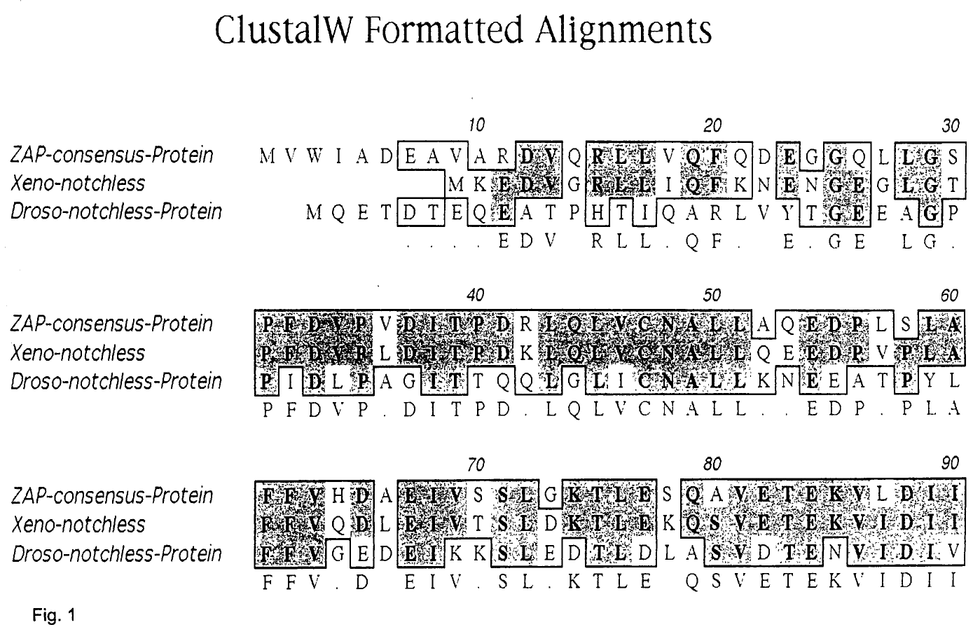

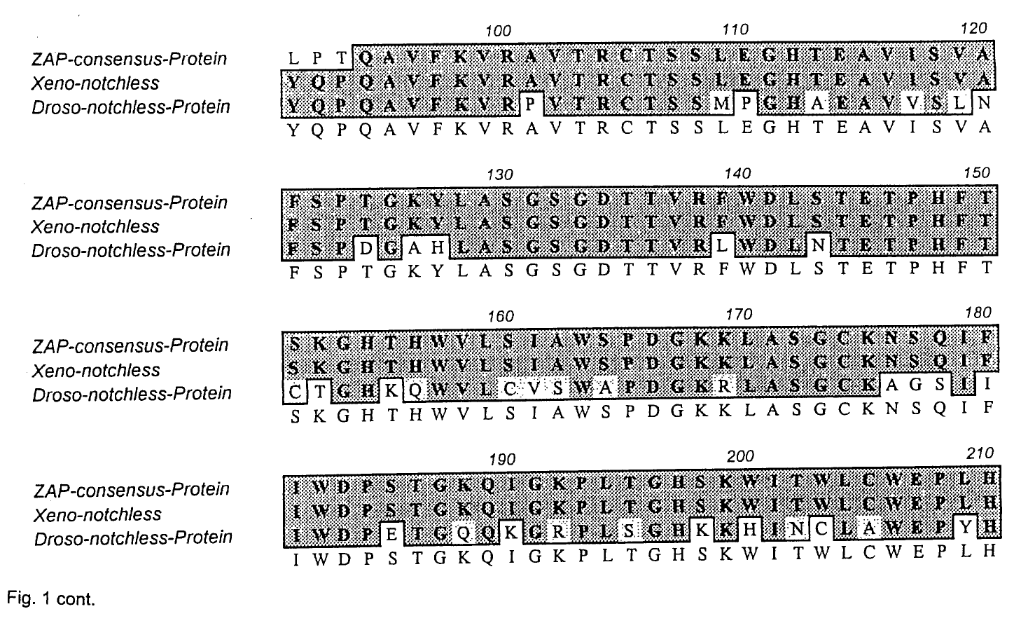

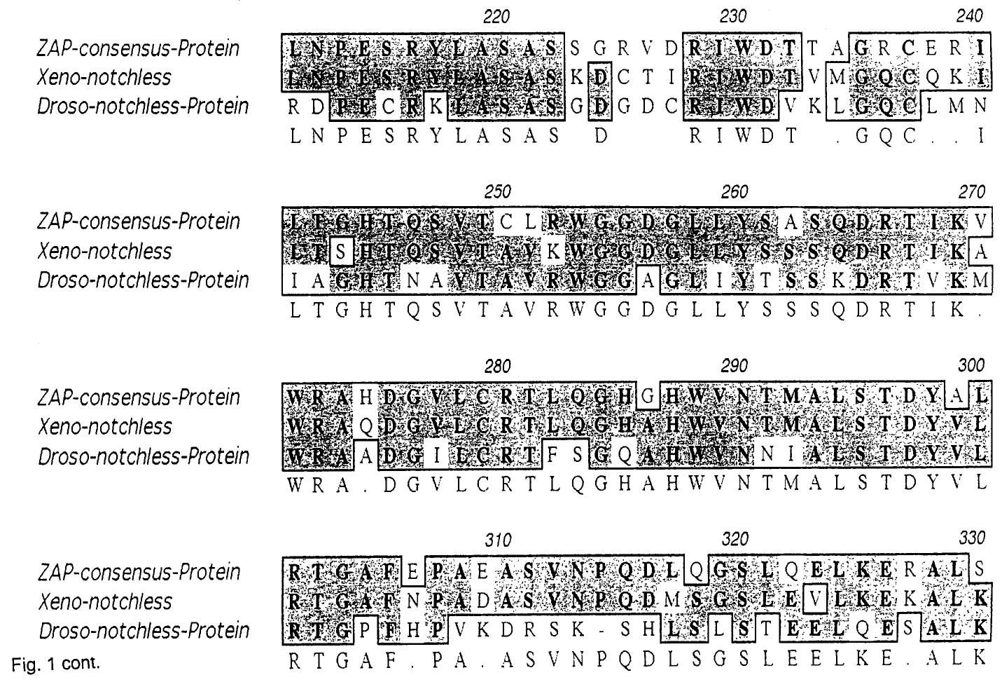

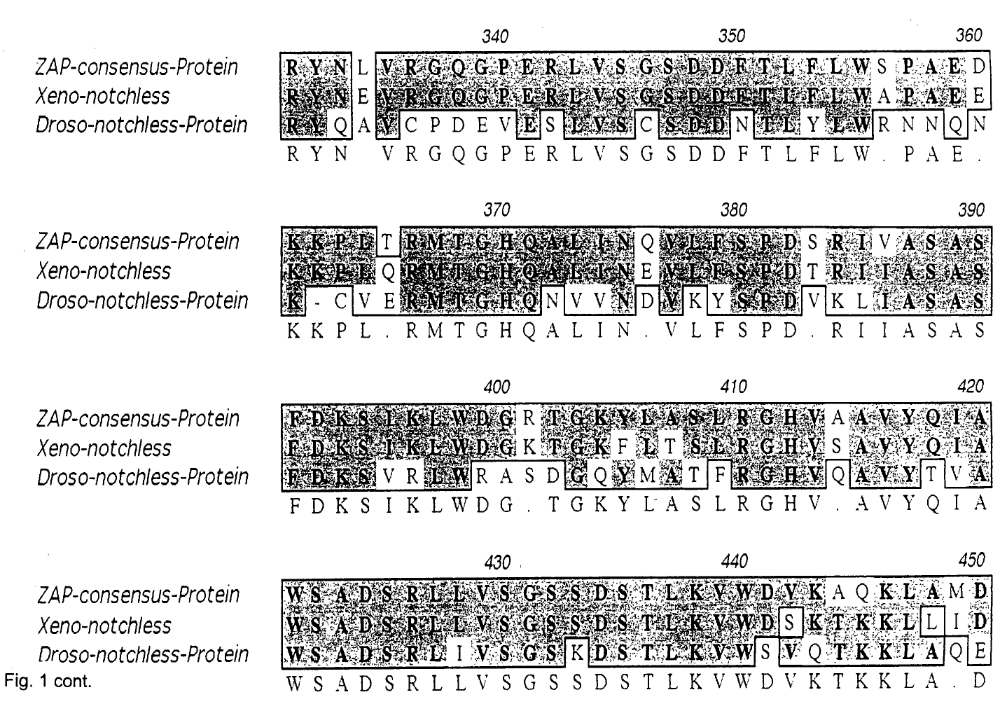

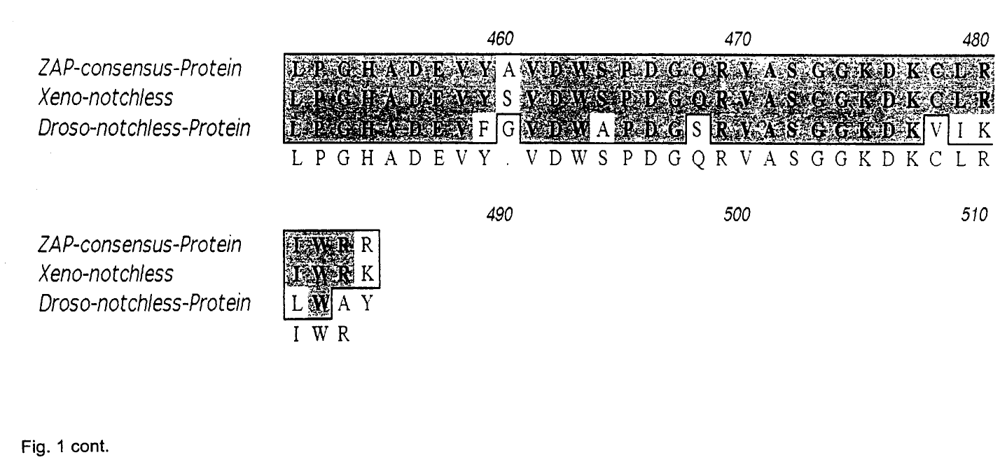

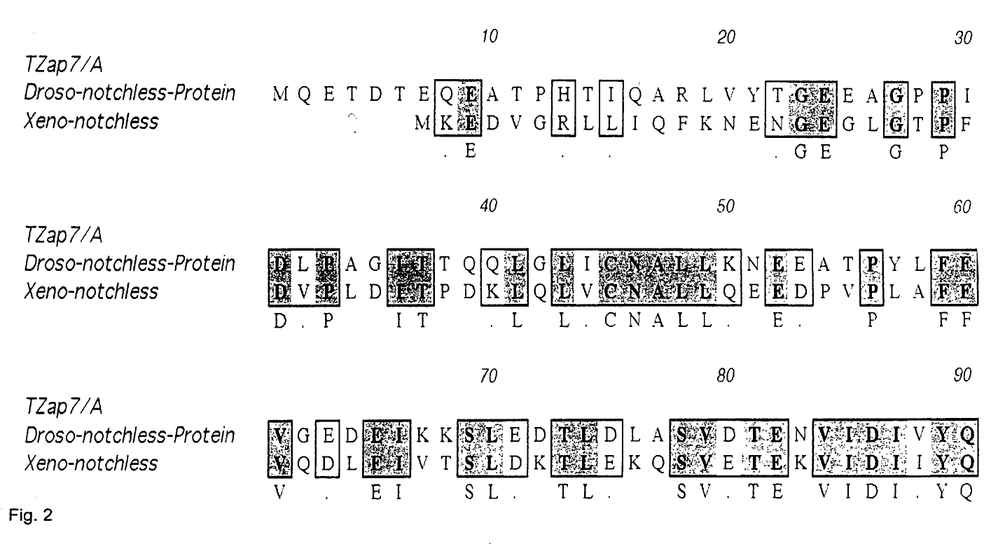

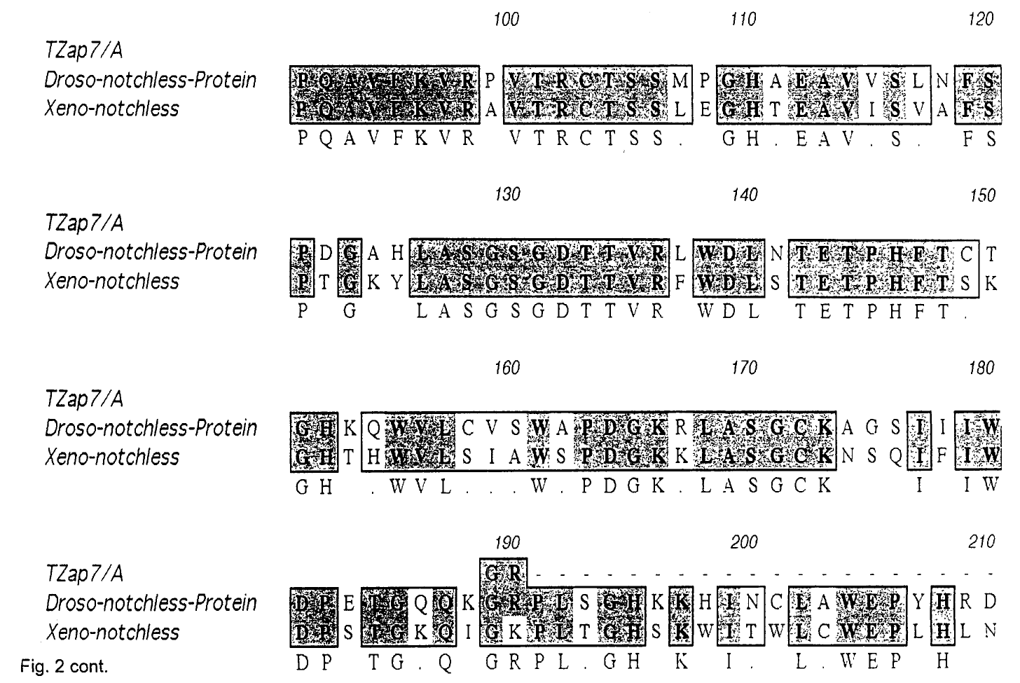

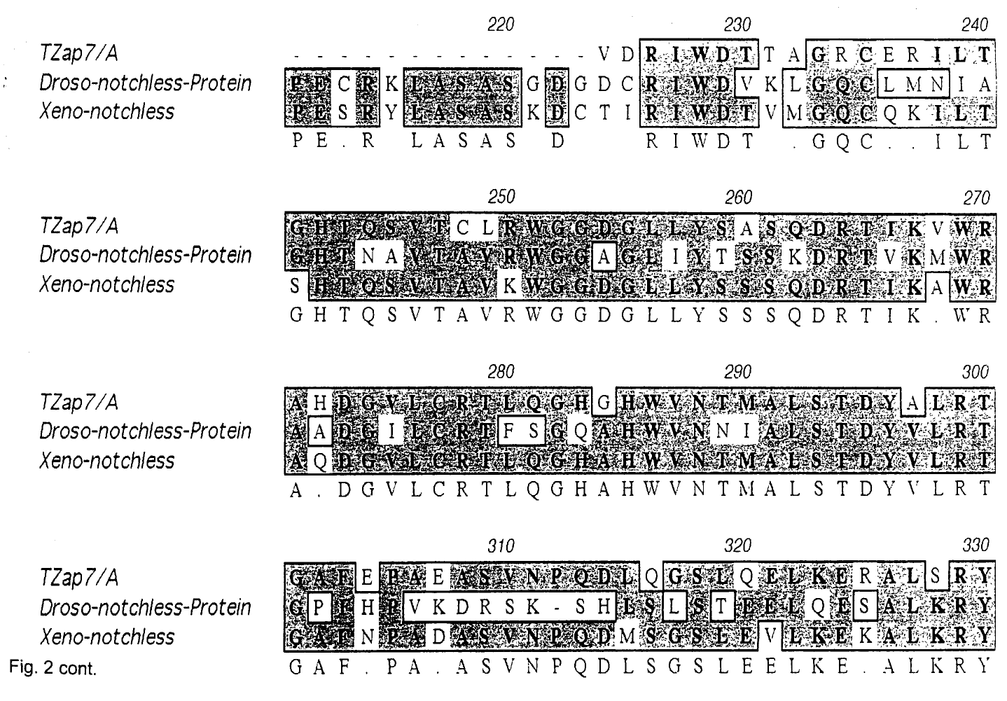

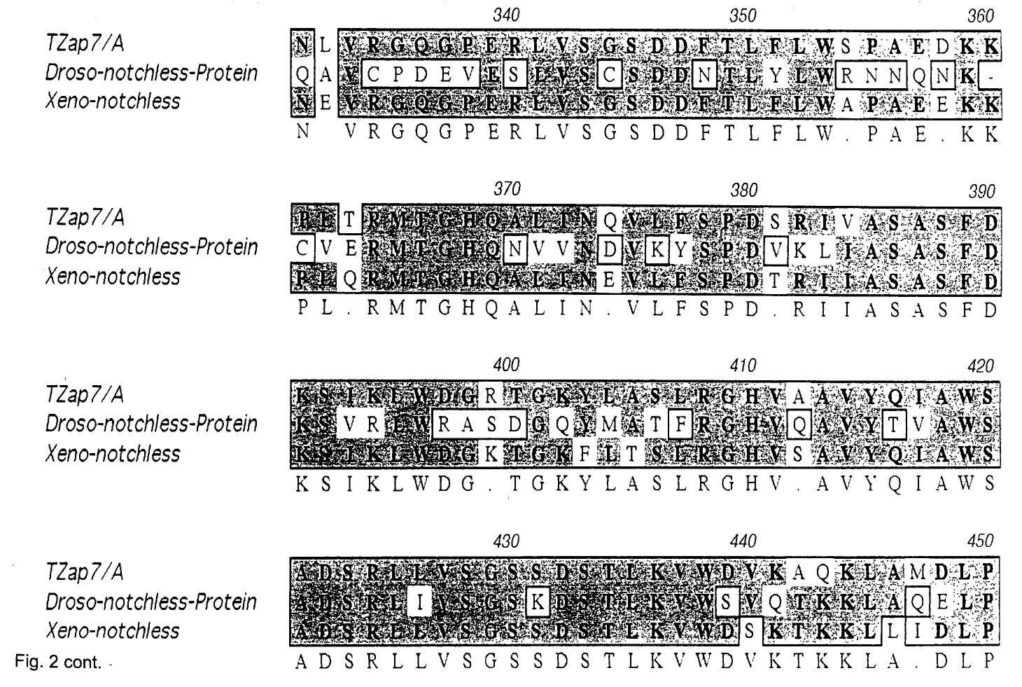

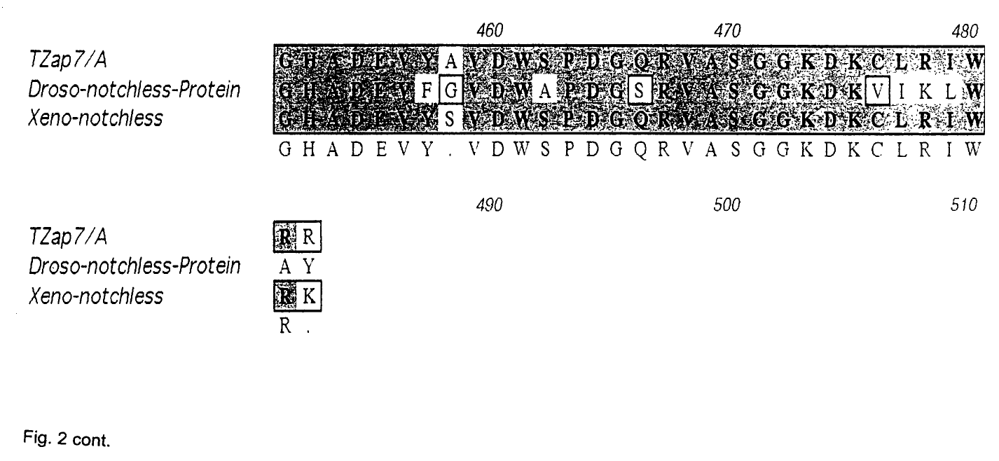

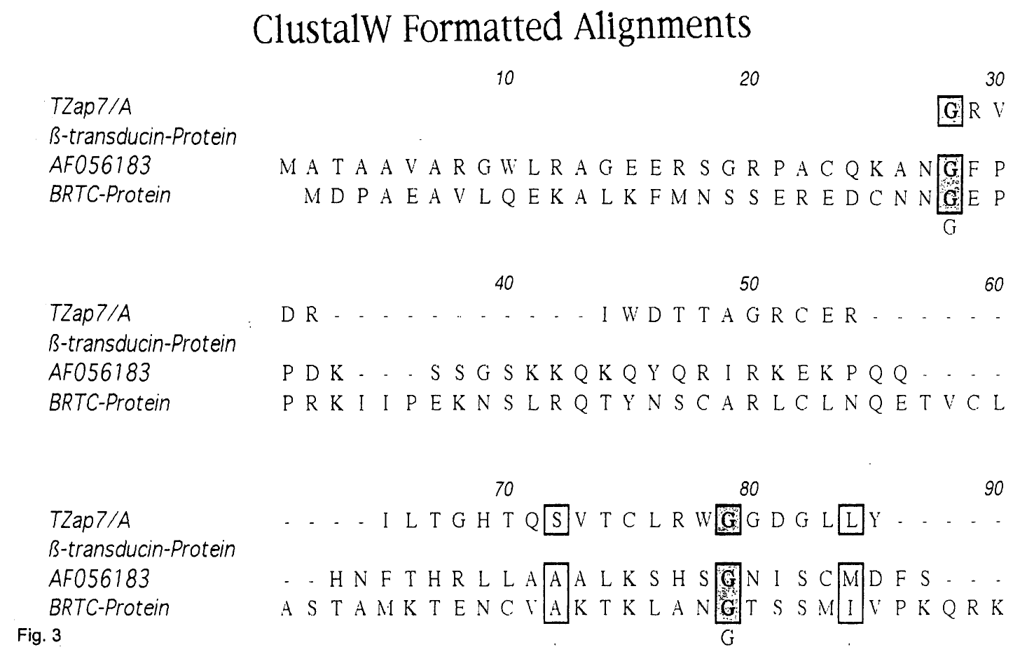

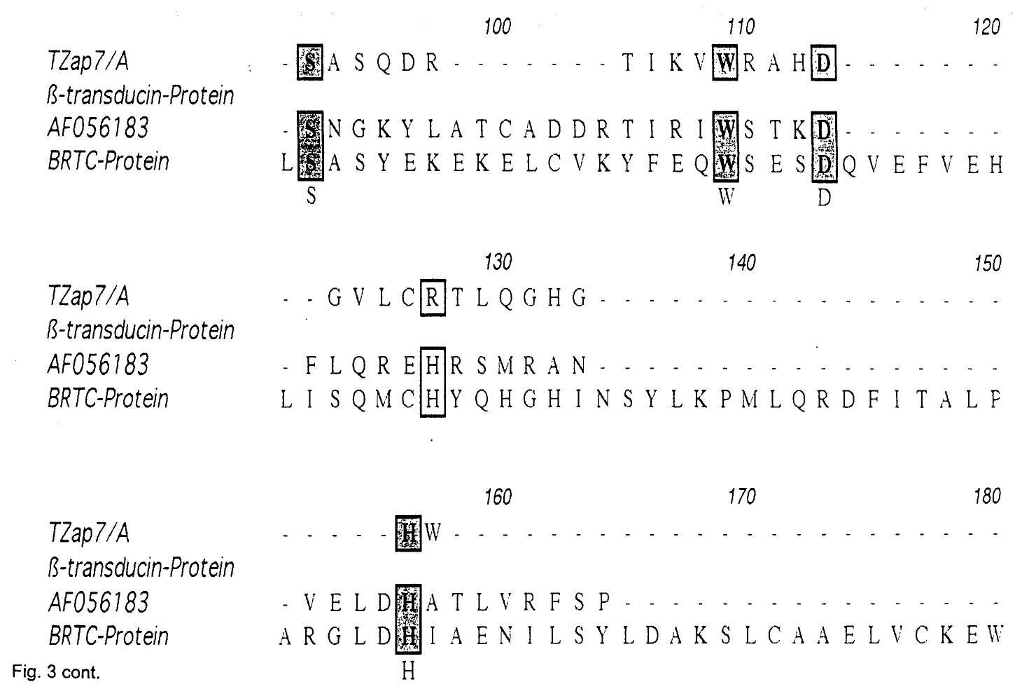

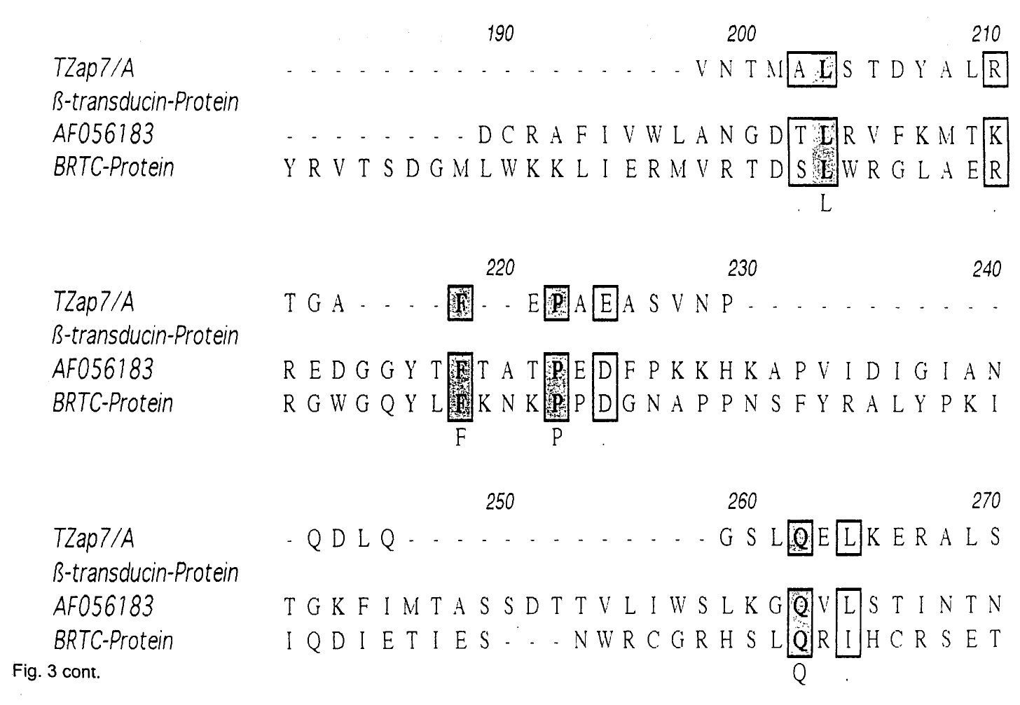

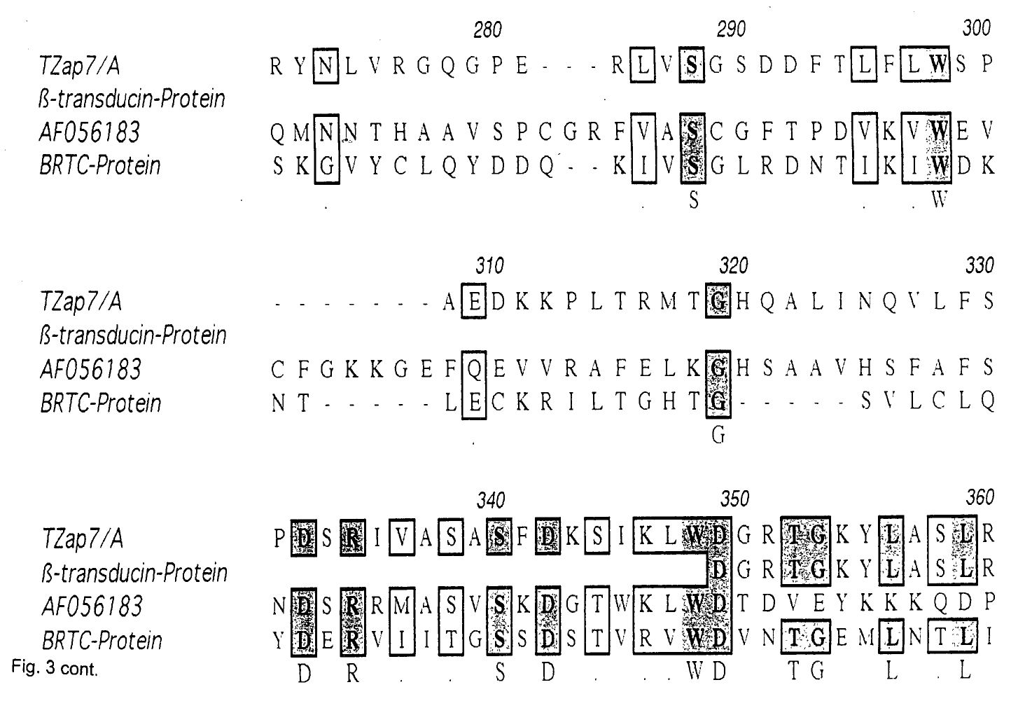

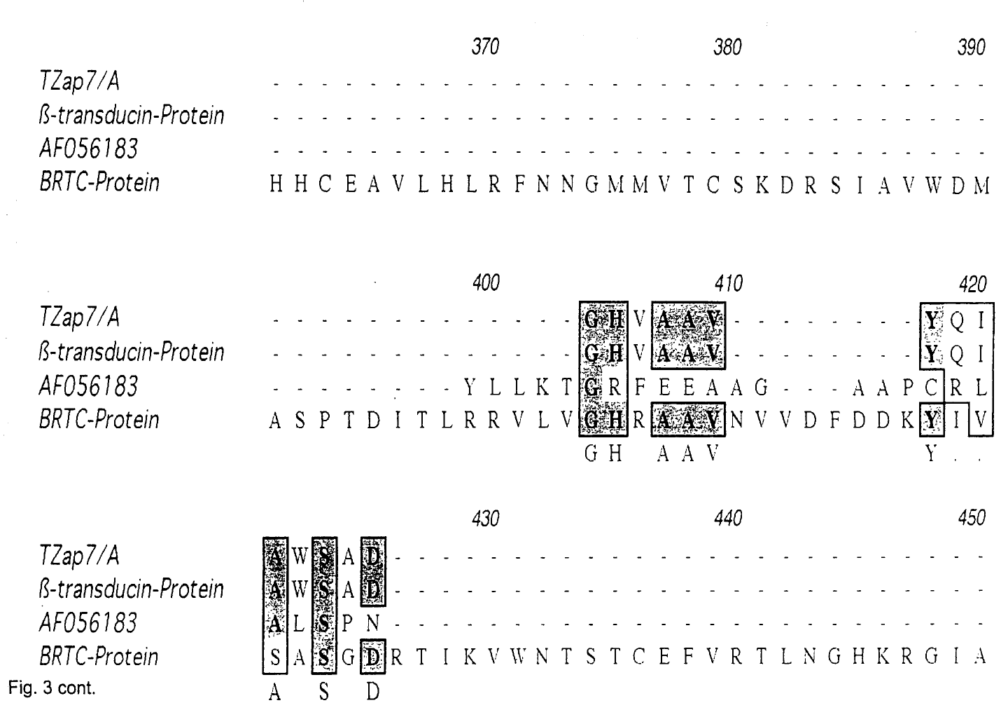

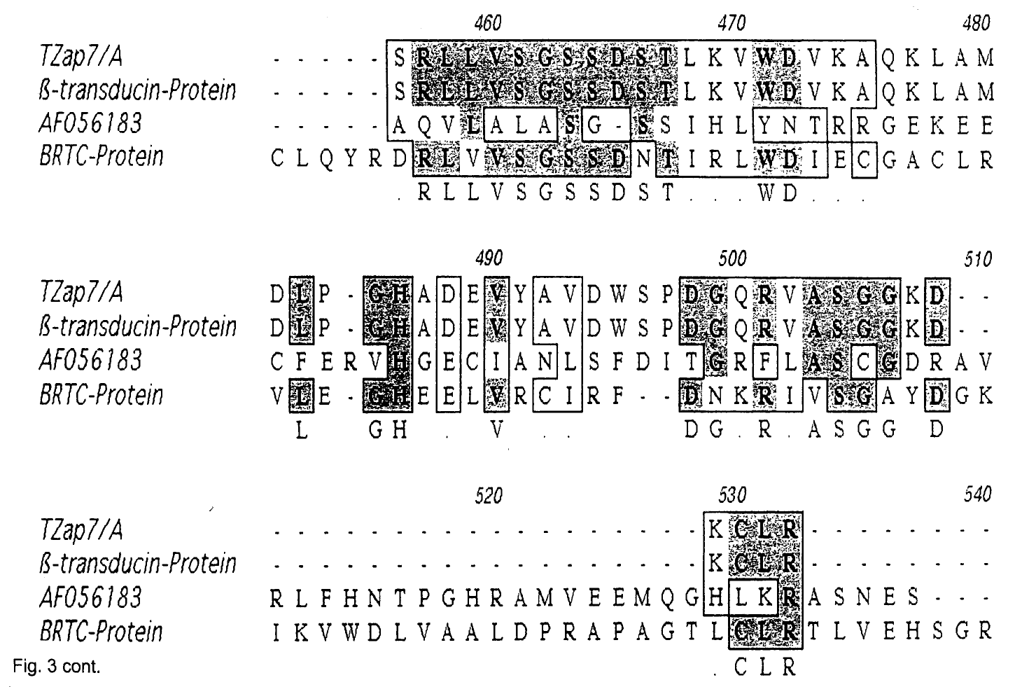

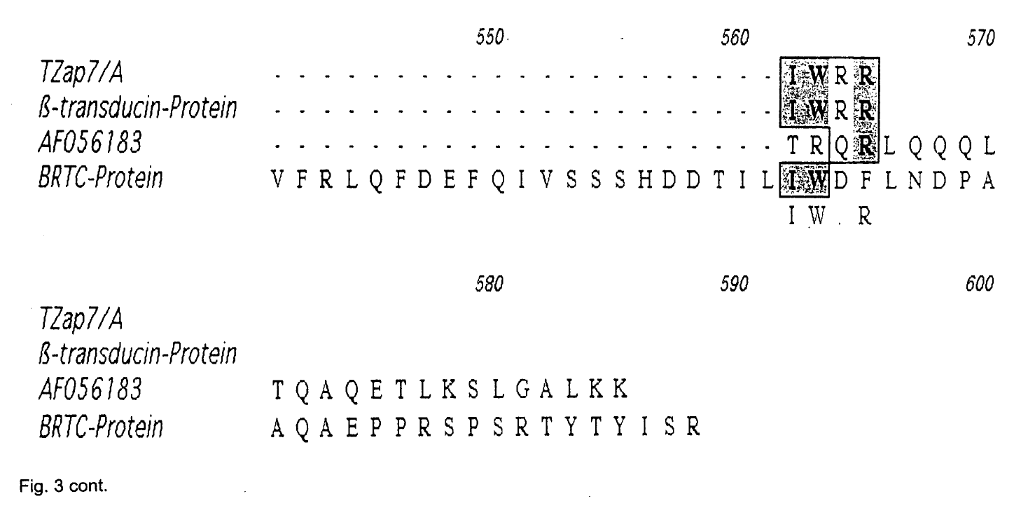

### DETAILED DESCRIPTION OF THE PRESENT INVENTION

In view of the need of therapeutic means for the diagnosis and treatment of diseases related to immune responses of the human body, the technical problem of the invention is to provide means and methods for the early detection and modulation of immune cell responses which are particularly useful in organ transplantation and autoimmune diseases. The solution to this technical problem is achieved by providing the embodiments characterized in the claims, namely novel immune response modulating proteins encoded by cell immune response cDNAs designated TZap7/A, TZap7/B and/or TZap7 are described which exhibit a central role in T cell activation. Thus, targeting of TZap7/A, TZap7/B and/or TZap7 protein and its encoding gene provides a novel therapeutic approach for modulation of the immune response. Accordingly, the invention relates to a polynucleotide encoding a TZap protein or a biologically active fragment thereof comprising a DNA sequence selected from the group consisting of:

(i) DNA sequences comprising a nucleotide sequence encoding the amino acid sequence depicted in SEQ ID NO. 2, 4 or 6;

(ii) DNA sequences comprising the nucleotide sequence depicted in SEQ ID NO. 1,3 or 5;

(iii) DNA sequences comprising a nucleotide sequence encoding a fragment or derivative of the protein encoded by the DNA sequence of (i) or (ii);

(iv) DNA sequences the complementary strand of which hybridizes with and which is at least 70% identical to the polynucleotide as defined in any one of (i) to (iii); and

(v) DNA sequences the nucleotide of which is degenerate to the nucleotide sequence of a DNA sequence of any one of (i) to (iv).

The terms “TZap7/A, TZap7/B and/or TZap7 protein”, and “TZap” are used interchangeably herein and in accordance with the present invention, denote a protein involved in the modulation of immune responses, e.g. modulating activation and differentiation of T cells. Studies which had been carried out within the scope of the present invention revealed that antisense polynucleotides directed to the mRNA encoding TZap protein are able to efficiently activate Notch, a class of proteins involved in signal transduction further explained in Example 1.

The term “leukocytes” generally denotes all kinds of white blood cells and preferably refers to monocytes and lymphocytes (B, T and NK cells), either in combination or individually. Thus, it should be understood that the term leukocyte may also be used herein so as to refer to individual species of leukocytes such as T cells only.

The term “biologically active fragment thereof” refers to peptides and polypeptides that are derived from said TZap7/A, TZap7/B and/or TZap7 protein and that are capable of effecting substantially the same or similar activity or at least one of said activities of TZap7/A (29 kDA), TZap7/B (53 kDA) and/or TZap7 (53 kDA).

In accordance with the present invention, novel genes induced in the early stage of T cell activation have been identified by examining mRNA expression in alloactivated human lymphocytes. Differential display-reverse transcription PCR analysis revealed a 230 bp cDNA fragment which was upregulated 24 h after allostimulation of a human T cell line and three corresponding cDNAs designated TZap7/A, TZap7/B and TZap7, respectively, have been cloned; see Example 1.

Expression studies with TZap7/A on mRNA of tissues from the human immune system revealed two bands of 1200 nt and 4000 nt, respectively. No differences in the expression level of human Nle in spleen, lymph Node, thymus, peripheral blood leukocytes, bone marrow and fetal liver could be detected. Most of these expression domains correspond to regions where Notch signaling has been implicated in cell fate specifications. Overexpression of notchless in the AKR1010 DP thymoma cell line leads to an activation of Notch resulting in a high level expression of TCR. Control cells do not exhibit detectable amounts of TCR on their surface.

Application of antisense RNA of TZap7/A to bone marrow and thymus of mice, resulted in an activation of Notch. In thymus this activation leads to a slight increase of CD8 T cells over CD4 T cells. In bone marrow, an increase of thymic-independent T cells was demonstrated, although this increase was not accompanied by a persistent block in B cell maturation, as it was shown for Notch overexpression in the bone marrow (Pui J C, Allman D, Xu L, DeRocco S, Karnell F G, Bakkour S, Lee J Y, Kadesch T, Hardy R R, Aster J C, Pear W S (1999): Notch1 expression in early lymphopoiesis influences B versus T lineage determination. Immunity, 11, 299-308).

TZap7/A, TZap7/B and TZap7 share homology with two different family of proteins which are involved in directing the immune response in different ways. Beta-transducin is one of the three subunits of the guanine nucleotide-binding proteins (G proteins) which act as intermediaries in the transduction of signals generated by transmembrane receptors (Neer E J, Schmidt C J, Nambudripad R, Smith T F (1994): The ancient regulatory-protein family of WD-repeat proteins. Nature, 371, 297-300 and Neer E J and Smith T F (1996) G protein heterodimers: new structures propel new questions. Cell, 84, 175-178).

In higher eukaryotes G-beta exists as a small multigene family of highly conserved proteins. They consist of eight tandem repeats of about 40 residues, each containing a central Trp-Asp motif which is called also WD-40 repeat. Beta-transducin repeat containing protein (HSBTRCP) has been shown to interact with HIV-1 Vpu which connects CD4 to the ER degradation pathway (Smith T F, Gaitatzes C G, Saxena K, Neer E J. (1999) The WD repeat: a common architecture for diverse functions. TIBS, 24, 181-185).

On one side, TZap7/A, TZap7/B and TZap7 share high percentage of identity on the nucleotide level within the 3′-region with beta-transducin family proteins supporting the hypothesis to be functionally involved in mediating the immune reaction during the course of response to an antigen. On the other side the same region is homologue to the notchless proteins in different species.

All these proteins have in common that they share WD-repeat motifs and are directly involved in early signalling cascade upon cell activation. Based on the homologies, it can be concluded that modulation of TZap by using antisense, small molecule, antibody as well as overexpression of the TZap proteins might lead to selected modulation of immune response and can serve as a novel target to treat diseases involving undesired immune reaction.

From the above it is evident that the nucleotide sequences depicted in SEQ ID No. 1, 3 and 5 encode novel immune response modulating proteins. By the provision of this nucleotide sequence it is now possible to isolate identical or similar polynucleotides which code for proteins with the biological activity of TZap7/A, TZap7/B and TZap7 from other species or organisms. Well-established approaches for the identification and isolation of such related sequences are, for example, the isolation from genomic or cDNA libraries using the complete or part of the disclosed sequence as a probe or the amplification of corresponding polynucleotides by polymerase chain reaction using specific primers.

Thus, the invention also relates to polynucleotides which hybridize to the above-described polynucleotides and differ at one or more positions in comparison to these as long as they encode a TZap7/A, TZap7/B or TZap7 protein as defined above. Such molecules comprise those which are changed, for example, by deletion(s), insertion(s), alteration(s) or any other modification known in the art in comparison to the above described polynucleotides either alone or in combination. Methods for introducing such modifications in the polynucleotides of the invention are well-known to the person skilled in the art; see, e.g., Sambrook et al. (Molecular cloning; A Laboratory Manual, Second Edition, Cold Spring Harbor Laboratory Press, Cold Spring Harbor N.Y. (1989)). The invention also relates to polynucleotides the nucleotide sequence of which differs from the nucleotide sequence of any of the above-described polynucleotides due to the degeneracy of the genetic code.

With respect to the DNA sequences characterized under (iv) above, the term “hybridizing” in this context is understood as referring to conventional hybridization conditions, preferably such as hybridization in 50% formamide/6×SSC/0.1% SDS/100 μg/ml ssDNA, in which temperatures for hybridization are above 37° C. and temperatures for washing in 0.1×SSC/0.1% SDS are above 55° C. Most preferably, the term “hybridizing” refers to stringent hybridization conditions, for example such as described in Sambrook, supra.

Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. “Stringent hybridization conditions” and “stringent wash conditions” in the context of nucleic acid hybridization experiments depend upon a number of different physical parameters. The most important parameters include temperature of hybridization, base composition of the nucleic acids, salt concentration and length of the nucleic acid. One having ordinary skill in the art knows how to vary these parameters to achieve a particular stringency of hybridization. In general, “stringent hybridization” is performed at about 25° C. below the thermal melting point (T m) for the specific DNA hybrid under a particular set of conditions. “Stringent washing” is performed at temperatures about 5° C. lower than the Tm for the specific DNA hybrid under a particular set of conditions. The Tm is the temperature at which 50% of the target sequence hybridizes to a perfectly matched probe. See Sambrook et al., page 9.51, hereby incorporated by reference.

The Tm for a particular DNA-DNA hybrid can be estimated by the formula:

Tm=81.5° C.+16.6(log10[Na+])+0.41(fraction G+C)−0.63(% formamide)−(600/l) where l is the length of the hybrid in base pairs.

The Tm for a particular RNA-RNA hybrid can be estimated by the formula:

Tm=79.8° C.+18.5(log10[Na+])+0.58(fraction G+C)+11.8(fraction G+C)2-0.35(% formamide)−(820/l).

TheTm for a particular RNA-DNA hybrid can be estimated by the formula:

Tm=79.8° C.+18.5(log10[Na+])+0.58(fraction G+C)+11.8(fraction G+C)2-0.50(% formamide)−(820/l).

In general, the Tm decreases by 1-1.5° C. for each 1% of mismatch between two nucleic acid sequences. Thus, one having ordinary skill in the art can alter hybridization and/or washing conditions to obtain sequences that have higher or lower degrees of sequence identity to the target nucleic acid. For instance, to obtain hybridizing nucleic acids that contain up to 10% mismatch from the target nucleic acid sequence, 10-15° C. would be subtracted from the calculated Tm of a perfectly matched hybrid, and then the hybridization and washing temperatures adjusted accordingly. Probe sequences may also hybridize specifically to duplex DNA under certain conditions to form triplex or other higher order DNA complexes. The preparation of such probes and suitable hybridization conditions are well known in the art.

An example of stringent hybridization conditions for hybridization of complementary nucleic acid sequences having more than 100 complementary residues on a filter in a Southern or Northern blot or for screening a library is 50% formamide/6× SSC at 42° C. for at least ten hours. Another example of stringent hybridization conditions is 6× SSC at 68° C. for at least ten hours. An example of low stringency hybridization conditions for hybridization of complementary nucleic acid sequences having more than 100 complementary residues on a filter in a Southern or northern blot or for screening a library is 6× SSC at 42° C. for at least ten hours. Hybridization conditions to identify nucleic acid sequences that are similar but not identical can be identified by experimentally changing the hybridization temperature from 68° C. to 42° C. while keeping the salt concentration constant (6× SSC), or keeping the hybridization temperature and salt concentration constant (e.g. 42° C. and 6× SSC) and varying the formamide concentration from 50% to 0%. Hybridization buffers may also include blocking agents to lower background. These agents are well-known in the art. See Sambrook et al., pages 8.46 and 9.46-9.58, herein incorporated by reference.

Wash conditions also can be altered to change stringency conditions. An example of stringent wash conditions is a 0.2× SSC wash at 65° C. for 15 minutes (see Sambrook et al., for SSC buffer). Often the high stringency wash is preceded by a low stringency wash to remove excess probe. An exemplary medium stringency wash for duplex DNA of more than 100 base pairs is 1× SSC at 45° C. for 15 minutes. An exemplary low stringency wash for such a duplex is 4× SSC at 40° C. for 15 minutes. In general, signal-to-noise ratio of 2× or higher than that observed for an unrelated probe in the particular hybridization assay indicates detection of a specific hybridization.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using NCBI BLASTx and BLASTn software. Alternatively, Fasta, a program in GCG Version 6.1. Fasta provides alignments and percent sequence identity of the regions of the best overlap between the query and search sequences (Pearson, 1990, herein incorporated by reference). For instance, percent sequence identity between nucleic acid sequences can be determined using Fasta with its default parameters (a word size of 6 and the NOPAMfactor for the scoring matrix) as provided in GCG Version 6.1, herein incorporated by reference.

Particularly preferred are polynucleotides which share 70%, preferably at least 85%, more preferably 90-95%, and most preferably 96-99% sequence identity with one of the above-mentioned polynucleotides and have the same biological activity. Such polynucleotides also comprise those which are altered, for example by nucleotide deletion(s), insertion(s), substitution(s), addition(s), and/or recombination(s) and/or any other modification(s) known in the art either alone or in combination in comparison to the above-described polynucleotides. Methods for introducing such modifications in the nucleotide sequence of the polynucleotide of the invention are well known to the person skilled in the art. Thus, the present invention encompasses any polynucleotide that can be derived from the above-described polynucleotides by way of genetic engineering and that encode upon expression a TZap7/A, TZap7/B and/or TZap7 protein or a biologically active fragment thereof.

It is also immediately evident to the person skilled in the art that regulatory sequences may be added to the polynucleotide of the invention. For example, promoters, transcriptional enhancers and/or sequences which allow for induced expression of the polynucleotide of the invention may be employed. A suitable inducible system is for example tetracycline-regulated gene expression as described, e.g., by Gossen and Bujard (Proc. Nati. Acad. Sci. USA 89 (1992), 5547-5551) and Gossen et al. (Trends Biotech. 12 (1994), 58-62).

In a further embodiment, the invention relates to nucleic acid molecules of at least 15 nucleotides in length hybridizing specifically with a polynucleotide as described above or with a complementary strand thereof. Specific hybridization occurs preferably under stringent conditions and implies no or very little cross-hybridization with nucleotide sequences encoding no or substantially different proteins. Such nucleic acid molecules may be used as probes and/or for the control of gene expression. Nucleic acid probe technology is well known to those skilled in the art who will readily appreciate that such probes may vary in length. Preferred are nucleic acid probes of 17 to 35 nucleotides in length. Of course, it may also be appropriate to use nucleic acids of up to 100 and more nucleotides in length. The nucleic acid probes of the invention are useful for various applications. On the one hand, they may be used as PCR primers for amplification of polynucleotides according to the invention. Another application is the use as a hybridization probe to identify polynucleotides hybridizing to the polynucleotides of the invention by homology screening of genomic DNA libraries. Nucleic acid molecules according to this preferred embodiment of the invention which are complementary to a polynucleotide as described above may also be used for repression of expression of a gene comprising such a polynucleotide, for example due to an antisense or triple helix effect or for the construction of appropriate ribozymes (see, e.g., EP-AL 0 291 533, EP-A1 0 321 201, EP-A2 0 360 257) which specifically cleave the (pre)-mRNA of a gene comprising a polynucleotide of the invention. Selection of appropriate target sites and corresponding ribozymes can be done as described for example in Steinecke, Ribozymes, Methods in Cell Biology 50, Galbraith et al. eds Academic Press, Inc. (1995), 449-460. Standard methods relating to antisense technology have also been described (Melani, Cancer Res. 51 (1991), 2897-2901). Furthermore, the person skilled in the art is well aware that it is also possible to label such a nucleic acid probe with an appropriate marker for specific applications, such as for the detection of the presence of a polynucleotide of the invention in a sample derived from an organism.

The above described nucleic acid molecules may either be DNA or RNA or a hybrid thereof. Furthermore, said nucleic acid molecule may contain, for example, thioester bonds and/or nucleotide analogues, commonly used in oligonucleotide anti-sense approaches. Said modifications may be useful for the stabilization of the nucleic acid molecule against endo- and/or exonucleases in the cell. Said nucleic acid molecules may be transcribed by an appropriate vector containing a chimeric gene which allows for the transcription of said nucleic acid molecule in the cell. Such nucleic acid molecules may further contain ribozyme sequences as described above.

In this respect, it is also to be understood that the polynucleotide of the invention can be used for “gene targeting” and/or “gene replacement”, for restoring a mutant gene or for creating a mutant gene via homologous recombination; see for example Mouellic, Proc. Natl. Acad. Sci. USA, 87 (1990), 47124716; Joyner, Gene Targeting, A Practical Approach, Oxford University Press.

In a preferred embodiment said nucleic acid molecules are labeled. Methods for the detection of nucleic acids are well known in the art, e.g., Southern and northern blotting, PCR or primer extension. In another preferred embodiment said nucleic acid molecules may be used for the suppression of TZap7/A, TZap7/B and/or TZap7 expression.

The polynucleotide of the invention encoding the above described TZap7/A, TZap7/B and/or TZap7 protein or biologically active fragments thereof may be, e.g., DNA, cDNA, RNA or synthetically produced DNA or RNA or a recombinantly produced chimeric nucleic acid molecule comprising any of those polynucleotides either alone or in combination. Preferably said polynucleotide is part of a vector. Such vectors may comprise further genes such as marker genes which allow for the selection of said vector in a suitable host cell and under suitable conditions. Preferably, the polynucleotide of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory-elements-permitting—expression in prokaryotic host cells comprise, e.g., the P_{L}, lac, trp or tac promoter in E. coli, and examples for regulatory elements permitting expression in eukaryotic host cells are the AOX1 or GALL promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription, such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. In this context, suitable expression vectors are known in the art such as Okayama-Berg cDNA expression vector pcDVl (Pharmacia), pCDM8, pRc/CMV, pcDNA1, pcDNA3 (In-vitrogene), or pSPORT1 (GIBCO BRL).

Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the protein of the invention may follow; see, e.g., the appended examples. In one preferred embodiment of the present invention antisense constructs are made based on the polynucleotide of the invention and combined with an appropriate expression control sequence.

In accordance with the above, the present invention relates to vectors, particularly plasmids, cosmids, viruses and bacteriophages used conventionally in genetic engineering that comprise a polynucleotide of the invention. Methods which are well known to those skilled in the art can be used to construct recombinant vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1989). Alternatively, the polynucleotides and vectors of the invention can be reconstituted into liposomes for delivery to target cells. The vectors containing the polynucleotides of the invention can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

In a still further embodiment, the present invention relates to a cell containing the polynucleotide or vector described above. Preferably, said cell is a eukaryotic, most preferably a mammalian cell if therapeutic uses of the protein are envisaged. Of course, yeast and less preferred prokaryotic, e.g., bacterial cells may serve as well, in particular if the produced protein is used as a diagnostic means. The polynucleotide or vector of the invention, which is present in the host cell, may either be integrated into the genome of the host cell or it may be maintained extrachromosomally.

The term “prokaryotic” is meant to include all bacteria which can be transformed or transfected with a DNA or RNA molecules for the expression of a protein of the invention. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells. Depending upon the host employed in a recombinant production procedure, the protein encoded by the polynucleotide of the present invention may be glycosylated or may be non-glycosylated. TZap7/A, TZap7/B and TZap7 proteins of the invention may also include an initial methionine amino acid residue. A polynucleotide of the invention can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989). The TZap7/A, TZap7/B and TZap7 proteins of the invention can be produced by recombinant DNA technology in -eukaryotic or prokaryotic-hosts. In general,-expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Furthermore, transgenic animals, preferably mammals, comprising cells of the invention may be used for the large scale production of the TZap7/A, TZap7/B and TZap7 proteins of the invention.

Alternatively, an animal, preferably mammalian, cell naturally having a polynucleotide of the invention present in its genome can be used and modified such that said cell expresses the endogenous gene corresponding to the polynucleotide of the invention under the control of an heterologous promoter. The introduction of the heterologous promoter which does not naturally control the expression of the polynucleotide of the invention can be done according to standard methods, see supra. Suitable promoters include those mentioned hereinbefore.

Thus, in a further embodiment, the present invention relates to a method for the production of a TZap7/A, TZap7/B and/or TZap7 protein or a biologically active fragment thereof comprising:

(a) culturing a host of the invention under conditions allowing for the expression of the protein; or

(b) in vitro translation of the polynucleotide of the invention;

and recovering the protein produced in (a) or (b).

The transformed hosts can be grown in fermentors and cultured according to techniques known in the art to achieve optimal cell growth. The TZap protein of the invention can then be isolated from the growth medium, cellular lysates, or cellular membrane fractions. Once expressed, the protein of the present invention can be purified according to standard procedures of the art, including ammonium sulfate precipitation, affinity columns, column chromatography, gel electrophoresis and the like; see, Scopes, “Protein Purification”, Springer-Verlag, N.Y. (1982). Substantially pure proteins of at least about 90 to 95% homogeneity are preferred, and 98 to 99% or more homogeneity are most preferred, for pharmaceutical uses. Once purified, partially or to homogeneity as desired, the proteins may then be used therapeutically (including extracorporeally) or in developing and performing assay procedures.

Hence, in a still further embodiment, the present invention relates to a TZap protein or a biologically active fragment thereof encoded by the polynucleotide of the invention or produced by a method of as described above. It will be apparent to those skilled in the art that the protein of the invention can be further coupled to other moieties as described above for, e.g., drug targeting and imaging applications. Such coupling may be conducted chemically after expression of the protein to site of attachment or the coupling product may be engineered into the protein of the invention at the DNA level. The DNAs are then expressed in a suitable host system, and the expressed proteins are collected and renatured, if necessary. Furthermore, the provision of the TZap7/A, TZap7/B and TZap7 proteins of the present invention enables the production of TZap specific antibodies. In this respect, hybridoma technology enables production of cell lines secreting antibody to essentially any desired substance that produces an immune response. RNA encoding the light and heavy chains of the immunoglobulin can then be obtained from the cytoplasm of the hybridoma. The 5′ end portion of the mRNA can be used to prepare cDNA to be inserted into an expression vector. The DNA encoding the antibody or its immunoglobulin chains can subsequently be expressed in cells, preferably mammalian cells.

Depending on the host cell, renaturation techniques may be required to attain proper conformation of the antibody. If necessary, point substitutions seeking to optimize binding may be made in the DNA using conventional cassette mutagenesis or other protein engineering methodology such as is disclosed herein.

Thus, the present invention also relates to an antibody specifically recognizing the peptide or polypeptide of the invention.

In a still further embodiment, the present invention relates to a cell that has been modified to express a TZap protein or an antibody of the invention. This embodiment may be well suited for, e.g., restoring B and/or T cell responsiveness to an antigen, in particular if the antibody of the invention capable of stimulating T cell proliferation is expressed in a form suitable to be presented on the cell surface. Moreover, the present invention relates to pharmaceutical compositions comprising any one of the above described polynucleotides, vectors, cells, proteins and/or antibodies and/or a ligand capable of binding to the TZap7/A, TZap7/B and/or TZap7 protein of the invention.

The antibody or ligand comprised in the pharmaceutical composition of the invention preferably has a specificity at least substantially identical to the binding specificity of the natural ligand of the TZap7/A, TZap7/B and/or TZap7 protein of the invention. Such an antibody or ligand can have a binding affinity of at least 10⁵M⁻¹, preferably higher than 10⁷M⁻¹ if leukocyte stimulation is envisaged and advantageously up to 10⁷M⁻¹ in case leukocyte suppression should be mediated.

In a preferred embodiment of the invention, said antibody is a monoclonal antibody, a polyclonal antibody, a single chain antibody, humanized antibody, or fragment thereof that specifically binds said peptide or polypeptide also including bispecific antibody, synthetic antibody, antibody fragment, such as Fab, Fv or scFv fragments etc., or a chemically modified derivative of any of these. Monoclonal antibodies can be prepared, for example, by the techniques as originally described in Kohler and Milstein, Nature 256 (1975), 495, and Galfrë, Meth. Enzymol. 73 (1981), 3, which comprise the fusion of mouse myeloma cells to spleen cells derived from immunized mammals with modifications developed by the art. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988. When derivatives of said antibodies are obtained by the phage display technique, surface plasmon resonance as employed in the BlAcore system can be used to increase the efficiency of phage antibodies which bind to an epitope of the peptide or polypeptide of the invention (Schier, Human Antibodies Hybridomas 7 (1996), 97-105; Malmborg, J. Immunol. Methods 183 (1995), 7-13). The production of chimeric antibodies is described, for example, in WO89/09622. Methods for the production of humanized antibodies are described in, e.g., EP-A1 0 239 400 and WO90/07861. A further source of antibodies to be utilized in accordance with the present invention are so-called xenogenic antibodies. The general principle for the production of xenogenic antibodies such as human antibodies in mice is described in, e.g., WO 91/10741, WO 94/02602, WO 96/34096 and WO 96/33735. Antibodies to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning A Laboratory Manual, Cold Spring Harbor Laboratory (1989) N.Y.

The pharmaceutical composition of the present invention may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well-known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 10⁶ to 10¹² copies of the DNA molecule. The compositions of the invention may be administered locally or systemically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as T cell costimulatory molecules or cytokines known in the art, or their inhibitors or activators depending on the intended use of the pharmaceutical composition.

Furthermore, it is envisaged by the present invention that the various polynucleotides and vectors encoding the above described peptides or polypeptides are administered either alone or in any combination using standard vectors and/or gene delivery systems, and optionally together with a pharmaceutically acceptable carrier or excipient. For example, the polynucleotide of the invention can be used alone or as part of a vector to express the (poly)peptide of the invention in cells, for, e.g., gene therapy or diagnostics of diseases related to disorders of the immune system. The polynucleotides or vectors of the invention are introduced into the cells which in turn produce the (poly)peptide. Subsequent to administration, said polynucleotides or vectors may be stably integrated into the genome of the subject. On the other hand, viral vectors may be used which are specific for certain cells or tissues and persist in said cells. Suitable pharmaceutical carriers and excipients are well known in the art. The pharmaceutical compositions prepared according to the invention can be used for the prevention or treatment or delaying of different kinds of diseases, which are related to lymphocyte and/or monocyte related immunodeficiencies and malignancies.

In another embodiment the present invention relates to a diagnostic composition comprising any one of the above described proteins, antibodies, (poly)peptides, polynucleotides, vectors or cells, and optionally suitable means for detection. The (poly)peptides and antibodies described above are, for example, suited for use in immunoassays in which they can be utilized in liquid phase or bound to a solid phase carrier. Examples of immunoassays which can utilize said (poly)peptides are competitive and non-competitive immunoassays in either a direct or indirect format. Examples of such immunoassays are the radioimmunoassay (RIA), the sandwich (immunometric assay) and the Western blot assay. The (poly)peptides and antibodies can be bound to many different carriers and used to isolate cells specifically bound to said polypeptides. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention.

There are many different labels and methods of labeling known to those of ordinary skill in the art. Examples of the types of labels that can be used in the present invention include enzymes, radioisotopes, colloidal metals, fluorescent compounds, chemiluminescent compounds, and bioluminescent compounds.

Said diagnostic compositions may also be used for methods for detecting expression of a polynucleotide of the invention by detecting the presence of mRNA coding for a TZap protein which comprises obtaining mRNA from a cell and contacting the mRNA so obtained with a probe comprising a nucleic acid molecule of at least 15 nucleotides capable of specifically hybridizing with a polynucleotide of the invention under suitable hybridizing conditions (see also supra), detecting the presence of mRNA hybridized to the probe, and thereby detecting the expression of the TZap protein by the cell.

Furthermore, the invention comprises methods of detecting the presence of a TZap protein in a sample, for example, a cell sample, which comprises obtaining a cell sample from a subject, contacting said sample with one of the aforementioned antibodies under conditions permitting binding of the antibody to the TZap protein, and detecting the presence of the antibody so bound, for example, using immunoassay techniques such as radioimmunoassay or enzyme-immunoassay. Furthermore, one skilled in the art may specifically detect and distinguish polypeptides which are functional TZap proteins from mutated forms which have-lost-or altered their immunomodulating activity by using an antibody which either specifically recognizes a (poly)peptide which has TZap7/A, TZap7/B and/or TZap7 activity but does not recognize an inactive form thereof or which specifically recognizes an inactive form but not the corresponding polypeptide having TZap7/A, TZap7/B and/or TZap7 activity. The antibodies of the present invention may also be used in affinity chromatography for purifying the TZap proteins or above described (poly)peptides and isolating them from various sources.

The invention also encompasses a method for diagnosing in a subject a predisposition to a disorder associated with the expression of a TZap7/A, TZap7/B and/or TZap7 allele which comprises isolating DNA from victims of the disorder associated with the under-or over-expression of a TZap7/A, TZap7/B and/or TZap7 protein; digesting the isolated DNA with at least one restriction enzyme; electrophoretically separating the resulting DNA fragments on a sizing gel; contacting the resulting gel with a nucleic acid probe as described above capable of specifically hybridizing to DNA encoding a TZap protein and labeled with a detectable marker; detecting labeled bands on the gel which have hybridized to the labeled probe to create a band pattern specific to the DNA of victims of the disorder associated with the expression of a TZap protein; preparing the subject's DNA according to the above-mentioned steps to produce detectable labeled bands on a gel; and comparing the band pattern specific to the DNA of victims of the disorder associated with the expression of a TZap protein and the subject's DNA to determine whether the patterns are the same or different and to diagnose thereby predisposition to the disorder if the patterns are the same. The detectable markers of the present invention may be labeled with commonly employed radioactive labels, such as, for example, ³²p and ³⁵S, although other labels such as biotin or mercury as well as those described above may be employed as well.

Various methods well-known to the person skilled in the art may be used to label the detectable markers. For example, DNA sequences and RNA sequences may be labeled with ³²p or ³⁵S using the random primer method. Once a suitable detectable marker has been obtained, various methods well-known to the person skilled in the art may be employed for contacting the detectable marker with the sample of interest. For example, DNA-DNA, RNA-RNA and DNA-RNA hybridizations may be performed using standard procedures. Various methods for the detection of nucleic acids are well-known in the art, e.g., Southern and northern blotting, PCR, primer extension and the like. Furthermore, the mRNA, cRNA, cDNA or genomic DNA obtained from the subject may be sequenced to identify mutations which may be characteristic fingerprints of TZap mutations in disorders associated with the expression of TZap or mutated versions thereof. The present invention further comprises methods, wherein such a fingerprint may be generated by RFLPs of DNA or RNA obtained from the subject, optionally the DNA or RNA may be amplified prior to analysis, the methods of which are well known in the art. RNA fingerprints may be performed by, for example, digesting an RNA sample obtained from the subject with a suitable RNA-Enzyme, for example RNase T₁, RNase T₂ or the like or a ribozyme and, for example, electrophoretically separating and detecting the RNA fragments on PAGE as described above or in the appended examples.

Advantageously, the pharmaceutical composition of the invention is intended for use in organ transplantation, for the treatment of autoimmune, allergic or infectious diseases, or for the treatment of tumors. An example for the use of the pharmaceutical composition of the invention for improving allograft or xenograft tolerance is described with respect to administration of an LFA-3 and CD2 binding protein, respectively, in WO93/06852.

In another embodiment, the present invention relates to a pharmaceutical composition comprising an agent which modulates immune response through the TZap7/A, TZap7/B and/or TZap7 protein of the invention, and optionally a pharmaceutically acceptable carrier. As is immediately evident to the person skilled in the art, the provision of the novel TZap7/A, TZap7/B and TZap7 proteins of the invention opens up the way of alternative approaches for modulating immune responses and treating corresponding diseases. The agent that, for example, stimulates the proliferation and/or differentiation of leukocytes through the TZap7/A, TZap7/B and/or TZap7 protein is expected to markedly enhance the proliferation of, e.g., (activated) T cells and thus is capable of augmenting the immune response. Examples for this type of “Vaccine” is described, e.g., in WO91/11194 and in the literature, e.g., referred to above. The agents to be employed in accordance with the present invention usually specifically bind to TZap7/A, TZap7/B and/or TZap7 protein in order to exert their effect. Such agents can be identified in accordance with a method of the invention described below. Such agents also comprise promoters-which can be inserted in front of the coding region of the TZap protein encoding gene, e.g., via gene transfer and homologous recombination in the 5′ untranslated region of the gene, see also supra. Such promoter may be regulated and thus permit the controlled expression of the TZap protein in certain cells.

In a further aspect the present invention relates to a method for identifying a binding partner to a TZap7/A, TZap7/B and/or TZap7 polypeptide comprising:

(a) contacting a TZap7/A, TZap7/B and/or TZap7 polypeptide of the invention with a compound to be screened; and

(b) determining whether the compound affects an activity of the polypeptide.

TZap7/A, TZap7/B and TZap7 polypeptides may be used to screen for molecules that bind to TZap7/A, TZap7/B and/or TZap7 or for molecules to which TZap7/A, TZap7/B and/or TZap7 binds. The binding of TZap7/A, TZap7/B and/or TZap7 and the molecule may activate (agonist), increase, inhibit (antagonist), or decrease activity of the TZap7/A, TZap7/B and/or TZap7 or the molecule bound. Examples of such molecules include antibodies, oligonucleotides, proteins (e.g., receptors), or small molecules.

Preferably, the molecule is closely related to the natural ligand of TZap, e.g., a fragment of the ligand, or a natural substrate, a ligand, a structural or functional mimetic; see, e.g., Coligan, Current Protocols in Immunology 1(2) (1991); Chapter 5. Similarly, the molecule can be closely related to the natural receptor to which TZap7/A, TZap7/B and/or TZap7 binds, or at least, a fragment of the receptor capable of being bound by TZap7/A, TZap7/B and/or TZap7 (e.g., active site). In either case, the molecule can be rationally designed using known techniques; see also infra.

Preferably, the screening for these molecules involves producing appropriate cells which express TZap, either as a secreted protein or on the cell membrane. Preferred cells include cells from mammals, yeast, Drosophila, or E. coli. Cells expressing TZap (or cell membrane containing the expressed polypeptide) are then preferably contacted with a test compound potentially containing the molecule to observe binding, stimulation, or inhibition of activity of either TZap or the molecule.

The assay may simply test binding of a candidate compound to TZap, wherein binding is detected by a label, or in an assay involving competition with a labeled competitor. Further, the assay may test whether the candidate compound results in a signal generated by binding to TZap.

Alternatively, the assay can be carried out using cell-free preparations, polypeptide/molecule affixed to a solid support, chemical libraries, or natural product mixtures. The assay may also simply comprise the steps of mixing a candidate compound with a solution containing TZap7/A, TZap7/B and/or TZap7, measuring TZap/molecule activity or binding, and comparing the TZap/molecule activity or binding to a standard.

Preferably, an ELISA assay can measure TZap level or activity in a sample (e.g., biological sample) using a monoclonal or polyclonal antibody. The antibody can measure TZap level or activity by either binding, directly or indirectly, to TZap or by competing with TZap for a substrate.

All of these above assays can be used as diagnostic or prognostic markers. The molecules discovered using these assays can be used to treat disease or to bring about a particular result in a patient (e.g., increase of immune response) by activating or inhibiting the TZap/molecule. Moreover, the assays can discover agents which may inhibit or enhance the production of TZap from suitably manipulated cells or tissues.

Therefore, the invention includes a method of identifying compounds which bind to TZap7/A, TZap7/B and/or TZap7 comprising the steps of:

(a) incubating a candidate binding compound with TZap; and

(b) determining if binding has occurred.

Moreover, the invention includes a method of identifying modulators of immune responses comprising the steps of:

(a) incubating a candidate compound with TZap7/A, TZap7/B and/or TZap7;

(b) assaying a biological activity as described above, and

(c) determining if a biological activity of TZap has been altered.

As mentioned hereinbefore, the polynucleotides and polypeptides of the present invention provide a basis-for the-development of mimetic- compounds that may be modulators of TZap or their encoding genes. It will be appreciated that the present invention also provides cell based screening methods that allow a high-throughput-screening (HTS) of compounds that may be candidates for such modulators.

Furthermore, the invention relates to a method for identifying leukocyte activating or co-stimulating compounds or for identifying inhibitors of leukocyte activation and stimulation comprising

(a) culturing T cells in the presence of the TZap7/A, TZap7/B and/or TZap7 protein, (poly)peptide, antibody, or cell described above and, optionally, in the presence of a component capable of providing a detectable signal in response to leukocyte activation, with a compound to be screened under conditions permitting interaction of the compound with the TZap protein, (poly)peptide, antibody or cell(s); and

(b) detecting the presence or absence of a signal generated from the interaction of the compound with the cells.

The term “compound” in the method of the invention includes a single substance or a plurality of substances which may or may not be identical.

Said compound(s) may be comprised in, for example, samples, e.g., cell extracts from, e.g., plants, animals or microorganisms. Furthermore, said compounds may be known in the art but hitherto not known to be capable of inhibiting proliferation of leukocytes or not known to be useful as an immune response costimulatory or modulating factor, respectively. The plurality of compounds may be, e.g., added to a simple in vitro, to the culture medium or injected into the cell.

If a sample containing (a) compound(s) is identified in the method of the invention, then it is either possible to isolate the compound from the original sample identified as containing the compound in question, or one can further subdivide the original sample, for example, if it consists of a plurality of different compounds, so as to reduce the number of different substances per sample and repeat the method with the subdivisions of the original sample. It can then be determined whether said sample or compound displays the desired properties by methods known in the art such as described herein and in the appended examples. Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. The methods of the present invention can be easily performed and designed by the person skilled in the art, for example in accordance with other cell based assays described in the prior art (see, e.g., EP-A-0 403 506) or by using and modifying the methods as described in the appended examples.

Furthermore, the person skilled in the art will readily recognize which further compounds and/or cells may be used in order to perform the methods of the invention, for example, B cells, interleukins, or enzymes, if necessary, that, e.g., convert a certain compound into the precursor which in turn stimulates or suppresses lymphocyte or monocyte activation or that provide for (co)stimulatory signals. Such adaptation of the method of the invention is well within the skill of the person skilled in the art and can be performed without undue experimentation.

Compounds which can be used in accordance with the method of the present invention include peptides, proteins, nucleic acids including cDNA expression libraries, antibodies, small organic compounds, ligands, peptidomimetics, PNAs and the like. Said compounds can also be functional derivatives or analogues of known B or T cell activators or inhibitors. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, N.Y., USA. Furthermore, said derivatives and analogues can be tested for their effects according to methods known in the art or as described, for example, in the appended examples.

Furthermore, peptidomimetics and/or computer aided design of appropriate activators or inhibitors of T cell activation can be used, for example, according to the methods described below. Appropriate computer programs can be used for the identification of interactive sites of a putative inhibitor and the TZap protein by computer assistant searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N.Y. Acad. Sci. 501 (1987), 1-13; Pabo, Biochemistry 25 (1986)5987-5991. The results obtained from the above-described computer analysis can be used in combination with the method of the invention for, e.g., optimizing known leukocyte activators or inhibitors. Appropriate peptidomimetics can also be identified by the synthesis of peptidomimetic combinatorial libraries through successive chemical modification and testing the resulting compounds, e.g., according to the methods described herein and in the appended examples. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of inhibitors or activators of leukocyte stimulation can be used for the design of peptidomimetic inhibitors or activators of leukocyte activation to be tested in the method of the invention (Rose, Biochemistry 35 (1996), 12933-12944; Rutenber, Bioorg. Med. Chem. 4 (1996), 1545-1558). Furthermore, ligands of Notch proteins described in Example 1 can be used as the starting material for any one of the above described methods.

In summary, the present invention provides methods for identifying compounds which are capable of modulating immune responses. Accordingly compounds identified in accordance with the method of the present invention to be inhibitors and activators, respectively, of immune response are also within the scope of the present invention.

Compounds found to enhance leukocyte proliferation may be used in the treatment of cancer or infections and related diseases. In addition, it may also be possible to specifically inhibit viral diseases, thereby preventing viral infection or viral spread. Compounds identified as suppressors of leukocyte proliferation can be used, e.g., for treating skin conditions (see, e.g., WO93/06866) or in allogenic or xenogenic cell or organ transplantation in order to avoid graft rejection; see also supra.

The compounds identified or obtained according to the method of the present invention are thus expected to be very useful in diagnostic and in particular for therapeutic applications.

Hence, in a further embodiment the invention relates to a method for the production of a pharmaceutical composition comprising formulating and optionally synthesizing the compound identified in step (b) or (c) of the above described methods of the invention in a pharmaceutically acceptable form. Hence, the present invention generally relates to a method of making a therapeutic agent comprising synthesizing the proteins, (poly)peptides, polynucleotides, vectors, antibodies or compounds according to the invention in an amount sufficient to provide said agent in a therapeutically effective amount to the patient. Methods for synthesizing these agents are well known in the art and are described, e.g. above.

The therapeutically useful compounds identified according to the method of the invention may be administered to a patient by any appropriate method for the particular compound, e.g., orally, intravenously, parenterally, transdermally, transmucosally, or by surgery or implantation (e.g., with the compound being in the form of a solid or semi-solid biologically compatible and resorbable matrix) at or near the site where the effect of the compound is desired. Therapeutic doses are determined to be appropriate by one skilled in the art, see also supra.

Such useful compounds can be, for- example, transacting factors which bind to the TZap protein of the invention. Identification of transacting factors can be carried out using standard methods in the art (see, e.g., Sambrook, supra, and Ausubel, supra). To determine whether a protein binds to the TZap7/A, TZap7/B and/or TZap7 protein of the invention, standard native gel-shift analyses can be carried out. In order to identify a transacting factor which binds to the TZap7/A, TZap7/B and/or TZap7 of the invention, the polypeptides and peptides of the invention can be used as an affinity reagent in standard protein purification methods, or as a probe for screening an expression library. Once the transacting factor is identified, modulation of its binding to the TZap protein of the invention can be pursued, beginning with, for example, screening for inhibitors against the binding of the transacting factor to the TZap protein of the present invention. Activation or repression of TZap specific genes could then be achieved in subjects by applying the transacting factor (or its inhibitor) or the gene encoding it, e.g., in a vector described in the embodiments hereinbefore. In addition, if the active form of the transacting factor is a dimer, dominant-negative mutants of the transacting factor could be made in order to inhibit its activity. Furthermore, upon identification of the transacting factor, further components- in the pathway leading to activation (e.g. signal transduction) or repression of a gene encoding the TZap protein of the present invention can then be identified. Modulation of the activities of these components can then be pursued, in order to develop additional drugs and methods for modulating the expression or activity of the TZap protein of the present invention.

Beside the above described possibilities to use the polynucleotides according to the invention for gene therapy and their use to identify homologous molecules, the described polynucleotides may also be used for several other applications, for example, for the identification of nucleic acid molecules which encode proteins which interact with the TZap7/A, TZap7/B and/or TZap7 protein described above. This can be achieved by assays well known in the art, for example, as described in Scofield (Science 274 (1996), 2063-2065) by use of the so-called yeast “two-hybrid system”. In this system the (poly)peptide encoded by the polynucleotides according to the invention or a smaller part thereof is linked to the DNA-binding domain of the GAL4 transcription factor. A yeast strain expressing this fusion protein and comprising a lacZ reporter gene driven by an appropriate promoter, which is recognized by the GAL4 transcription factor, is transformed with a library of cDNAs which will express animal, preferably mammal proteins or peptides thereof fused to an activation domain. Thus, if a peptide encoded by one of the cDNAs is able to interact with the fusion protein comprising a (poly)peptide of the invention, the complex is able to direct expression of the reporter gene. In this way the polynucleotide according to the invention and the encoded peptide can be used to identify peptides and proteins interacting with TZap proteins.

Other methods for identifying compounds which interact with the TZap protein according to the invention or nucleic acid molecules encoding such molecules are, for example, the in vitro screening with the phage display system as well as filter binding assays or “real time” measuring of interaction using, for example, the BIAcore apparatus (Pharmacia); see references cited supra.

Furthermore, the present invention relates to the use of the polynucleotide, the nucleic acid molecule, the vectors, peptides, polypeptides, antibodies and cells of the invention as well as compounds identified in accordance with a method of the invention described hereinabove for the preparation of a composition for diagnosing and/or the treatment of diseases involving T cell activation and associated with Th1 and Th2 immune response, for the treatment of acute and chronic rejection of allo-and xeno organ transplants and bone marrow transplantation, for the treatment of rheumatoid arthritis, lupus erythramatodes, multiple sclerosis, encephalitis, vasculitis, diabetes mellitus, pancreatitis, gastritis, thyroiditis, for the treatment of maligne disorders of T, B or NK cells, for the treatment of asthma, lepramatosis, Helicobacter pylori associated gastritis or for the treatment of skin tumors, adrenal tumors or lung tumors, wound healing, growth disorders, inflammatory and/or infectious diseases.

The polynucleotides, vectors, cells, proteins, (poly)peptides, antibodies, inhibitors, activators, pharmaceutical and diagnostic compositions, uses and methods of the invention can be used for the treatment of all kinds of diseases hitherto unknown as being related to or dependent on the modulation of TZap. The pharmaceutical compositions, methods and uses of the present invention may be desirably employed in humans, although animal treatment is also encompassed by the methods and uses described herein. Further methods and uses that can be employed in accordance with the present invention are described in WO99/11782 the disclosure of which is hereby specifically incorporated. These and other embodiments are disclosed and encompassed by the description and Examples of the present invention. Further literature concerning any one of the antibodies, methods, uses and compounds to be employed in accordance with the present invention may be retrieved from public libraries and databases, using for example electronic devices. For example the public database “Medline” may be utilized which is available on the Internet, for example under http://www.ncbi.nlm.nih.gov/PubMed/medline.html. Further databases and addresses, such as http://www.ncbi.nim.nih.gov/, http://www. infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

Throughout this specification and claims, the word “comprise,” or variations such as “comprises” or “comprising,” will be understood to imply the inclusion of a stated integer or group of integers but not the exclusion of any other integer or group of integers.

A better understanding of the present invention and of its many advantages will be had from the following examples, given by way of illustration.

### EXAMPLES

Isolation of cDNAs from Alloactivated Human T Cell Line Encoding Novel Proteins which Share Homology with Notchiess Proteins in Drosophila and Xenopus as well as with Proteins from the β-Transducin Family in Human Cells

To identify genes induced during the early phase of T cell activation in response to alloantigens, differential display analysis of mRNA expression was performed using an allostimulated human T cell line. Activation of human T cells was performed as follows. In accordance with the ethical standards as formulated in the Helsinki Declaration of 1975 peripheral blood was obtained from healthy volunteers. Lymphocytes (PBL) were isolated using standard Ficoll centrifugation and resuspended in RPMI containing 10% fetal calf serum. Responder PBL were stimulated with equal numbers of irradiated (3000 rad, 13 min) stimulator lymphocytes. To establish an alloactivated human T cell line, cells were co-cultured for 24 h in tissue flasks at an initial concentration of 10⁶ cells/ml. Responding human T cells were restimulated three times with the irradiated stimulator lymphocytes from the same volunteer at 10 day intervals. The third restimulation was carried out with 5×10⁶ cells/ml irradiated stimulator cells, and total RNA was isolated immediately and 24 h after co-culturing. The RNA expression pattern was analyzed immediately and 24 h after the third allostimulation. Differential display-reverse transcription PCR (DDRT-PCR) analysis was then performed. Total RNA was isolated from lymphocytes immediately and 24 h after co-culturing using RNAzol B (Tel-Test) and differential display was performed as described previously [10]. Briefly, 2 μg of total RNA was used for reverse transcription with an oligo d(T) primer (25 nM) and 200 U MMLV reverse transcriptase (Gibco BRL). 50 ng of the resulting cDNA was used for subsequent PCR with primers 5′-GATGCCACCATGG-3′ (SEQ ID NO: 7) and 5′-TGCGTCTGGTTCT-3′ (SEQ ID NO: 8) artificial DNA. PCR was performed in a 20 μl reaction containing 1.25 mM MgCl2, 50 mM KCl, 10 mM Tris HCl, pH 8.3, 20 nM dNTP, 2.5 nM of each primer, 5 μCi 35S[dATP], and 0.3 U Taq polymerase (Promega). PCR products were separated by electrophoresis in a 6% polyacrylamid-urea gel (Sequagel, National Diagnostics) and subjected to autoradiography.

Using the primer pair given above, differential display RT-PCR revealed a single 230 bp cDNA fragment, which was significantly increased 24 h after stimulation.

To obtain a full length cDNA, this fragment was used to screen a human leukocyte cDNA in λ Trip1Ex. The differentially expressed CDNA fragment was excised from the filter, eluted in 0.5 M ammonium acetate/1 mM EDTA, pH 8.3, and ethanol precipitated. The cDNA product was reamplified, electrophoresed in a 2% agarose gel and purified using Gene Clean kit (Quiagen). The recovered cDNA product was blunt-ended with Klenow enzyme (Gibco BRL) following standard protocols [11] and ligated into pBluescript SK⁺ vector. After labeling with α³²P[dCTP] (800 mCi/mmol, Amersham Inc.) using the random priming method [12], the cDNA fragment isolated from DDRT-PCR was used as a probe to screen a human leukocyte cDNA in λ Trip1Ex. Hybridization was carried out for 24 h at 42 C in 40% formamide, 10% Dextran sulfate, 4× SSC (1× SSC consists of 150 mM NaCl, 15 mM sodium citrate, pH 7.0), 0.8×Denhardt's solution (1×Denhardt's solution contains 0.02% polyvinylpyrrolidone, 0.02% Ficoll, 0.02% bovine serum albumin), 0.5% sodium dodecyl sulfate (SDS), and 20 μg/ml salmon sperm DNA. The filters were washed twice for 20 min with 2× SSC/10% SDS at room temperature, and for 30 min with 0.2× SSC/10% SDS at 65 C, followed by autoradiography. From 2×10⁶ recombinants, two positive clones were plaque purified, subcloned into pBluescript SK⁺ vector. Complete cDNA inserts were sequenced according to the method of Sanger [13], using a primer walking strategy starting from primers flanking the multiple cloning site of the plasmid. Sequence analysis was performed using Geneworks software system. For homology searches NCBl BLASTx and BLASTn software were used. Three clones, TZap7/A, TZap7/B and TZap7 were isolated and sequenced. Sequence analysis of TZap7/B revealed a 1928 bp cDNA (SEQ ID NO: 1) with an open reading frame of 1455 Nt, predicting a protein length of 485 amino acids (SEQ ID NO: 2).

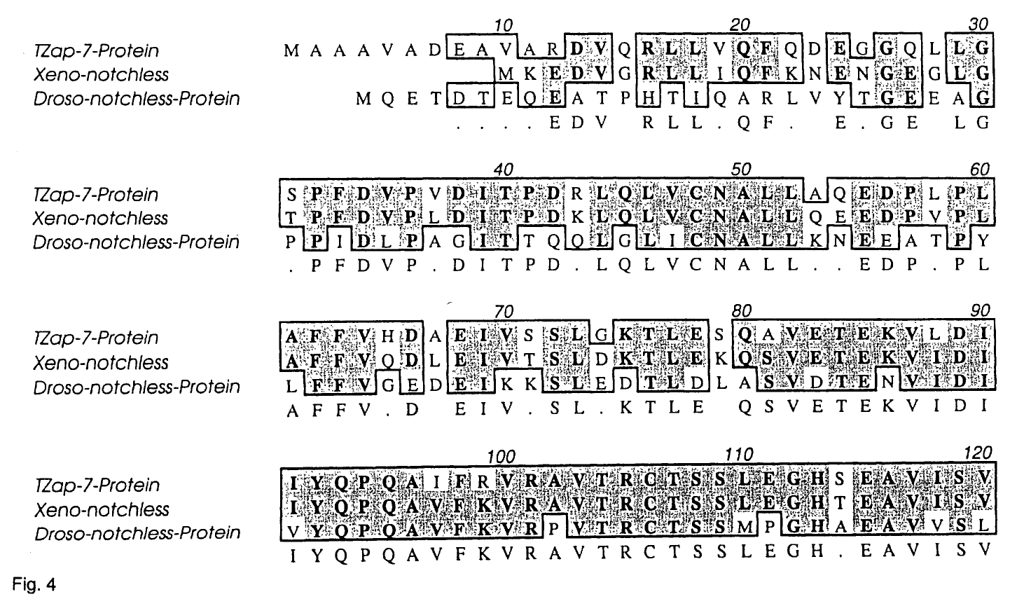

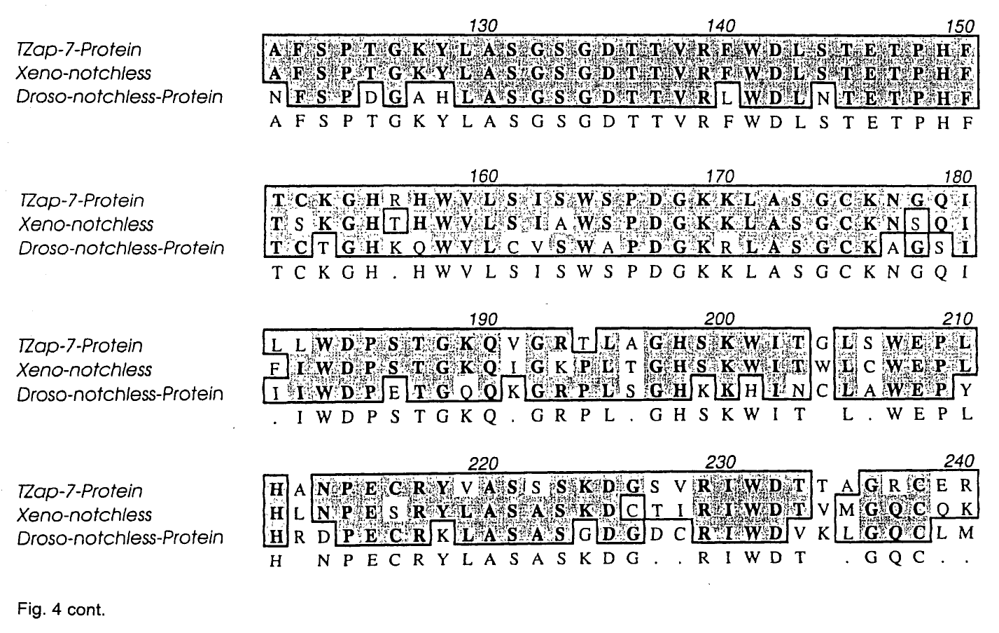

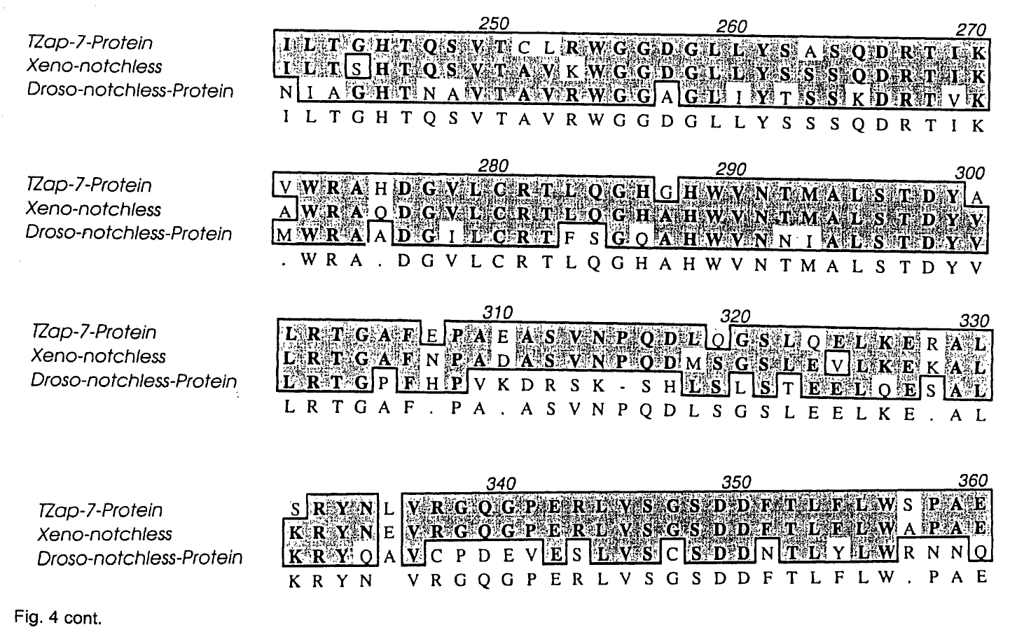

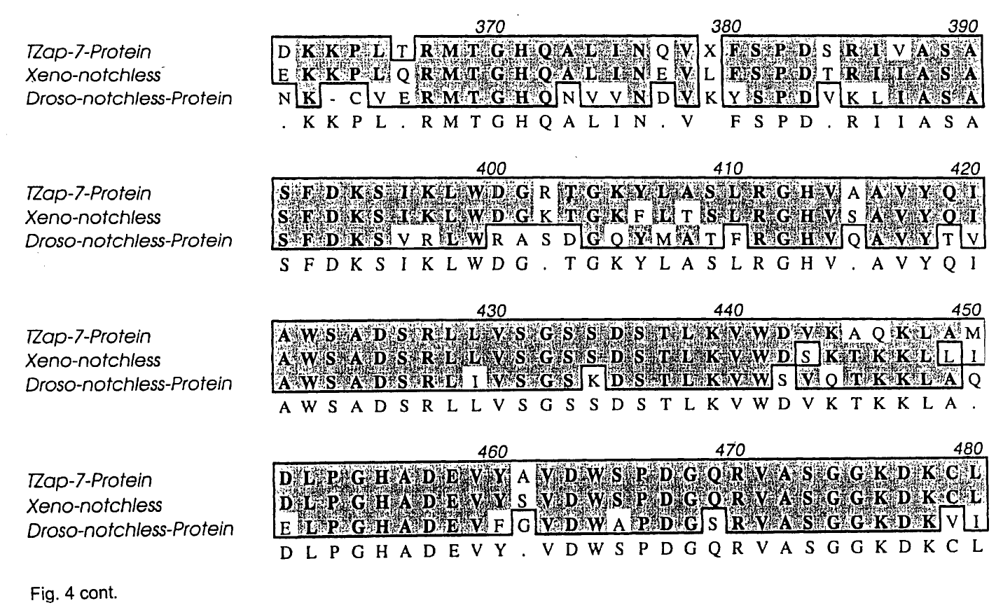

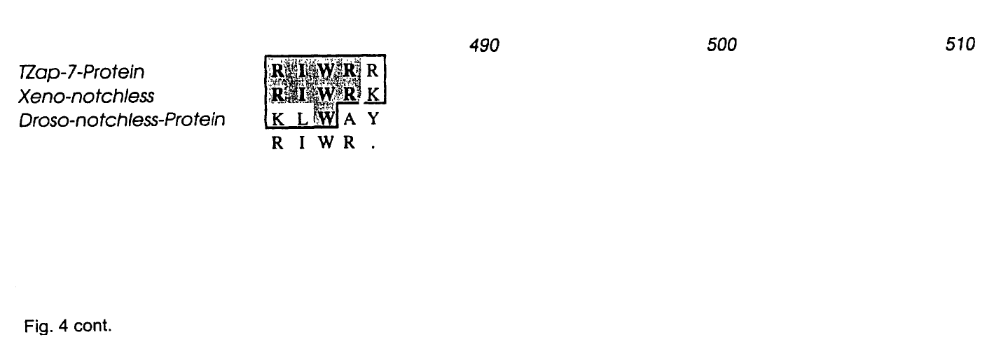

The putative translation product of TZap7/B shows extensive homology to Notchless proteins of Drosophila and Xenopus. Homologies are as high as 83% amino acids being identical between TZap and Xenopus Nle and 55% between TZap and Drosophila Nle. Sequencing of the second clone, designated TZap7/A, revealed a cDNA of 1170 bp (SEQ ID NO: 3) with an open reading frame of 786 Nt, predicting a protein length of 262 amino acids (SEQ ID NO: 4). The putative translation product of this clone also shows significant homologies to the Notchless proteins of Drosophila and Xenopus. Sequence identities ranging from 31% between Drosophila and TZap7/A and 45% between Xenopus and the human Nle. Furthermore, a third cDNA clone was obtained designated TZap7 with cDNA of 1,859 base pairs in length (SEQ ID NO: 5) encoding a protein of 458 amino acids in length (SEQ ID NO: 6). The amino acid sequence of TZap7 shows homology with notchless proteins similar to TZap7/B; see FIG. 4. The relationship of the novel TZap proteins and Notch, Notchless and transducin proteins will be discussed below.

Notch-Signalling in Differentiation

During development, a cell's fate is determined by a type of signaling known as lateral inhibition or lateral specification (Artavanis-Tsakonas, S., Matsuno, K. and Fortini, M. E. (1995) Notch Signalling. Science, 268, 225-232). Such signals are transmitted between cells in direct contact with each other and direct the differentiation of distinct cell types emerging from a group of cells that have otherwise equivalent potential. This evolutionarily conserved pathway is mediated by the transmembrane receptor protein encoded by the Notch gene of Drosophila and its vertebrate homologues, as well as by related proteins that are encoded by the lin-12 and glp-1 genes of C. elegans (Artavanis-Tsakonas, S., Matsuno, K. and Fortini, M. E. (1995) Notch Signalling. Science, 268, 225-232).

One of the best understood examples of Notch participation in local intercellular communication is the decision between so-called AC and VU cell fates in the nematode C. elegans (Greenwald. I. and Rubin, G. M. (1992) Making a difference: the role of cell-cell interactions in establishing separate identities for equivalent cells. Cell, 68, 271-281).

Each of two cells that are in direct contact with each other has the capacity to become an anchor cell (AC), but stochastically only one adopts the AC fate while the other adopts the ventral uterine precursor (VU) fate. It is thought that when one cell chooses its fate, it sends a signal to its neighbor, inhibiting the neighbor from choosing the same fate and directing it to assume an alternative fate. Lateral signaling or notch signaling appears to guide the development of numerous structures in invertebrates such as the proper formation of the nervous system, the mesoderm and the germ line in embryonic Drosophila as well as the larval Malpighian tubules, adult sensory bristles and eye structures of the fly (reviewed in Artavanis-Tsakonas et al., 1995).

The role of lateral inhibition is best studied in invertebrates but recent studies indicate an important role of notch signaling in numerous cell fate decisions in mammals.

Role of Notch Family Members in Mammalian Cell Fate Specification

The role of Notch family members in mammals was explored both through gene overexpression and gene inactivation. In mice and man, each of which possess at least four different Notch proteins, chromosomal rearrangments that affect Notch genes are associated with certain neoplasias. (Ellisen, L. W., Bird, J., West, D. C., Soreng, A. L., Reynolds, T. C., Smith, S. D. and Sklar, J. (1991): Tan-1, the human homolog of the Drosophila notch gene, is broken by chromosomal translocations in T lymphoblastic neoplasms. Cell, 66, 649-661). The vertebrate Notch genes are expressed throughout developing tissues at embryonic stages and in proliferative cell layers of mature tissues. Mice defective for one of their Notch genes die before 11.5 days of gestation with extensive regions of cell death (Swiatek, P. J., Lindsell, C. E., del Amo, F. F., Weinmaster, G. and Gridley, T. (1994): Notch1 is essential for postimplantation development in mice. Genes & Development, 6, 707-719). These findings imply an essential function of Notch for the development in mammals.

That Notch is involved in mammalian cell fate determination was suggested by constitutive activation of Notch1 in immature thymocytes that resulted in a biased CD4 versus CD8 lineage decision in favor of CD8 T cells as well as in T cell receptor expression that was skewed toward α/β TCRs (Robey, E., Chang, D., Itano, A., Cado, D., Alexander, H., Lans, D., Weinmaster, G. and Salmon, P. (1996): An activated form of notch influences the choice between CD4 and CD8 T cell lineage. Cell, 87, 483-492). And even earlier in T cell lineage, it was shown that constitutive expression of Notch1 results in a emergence of a population of thymic-independent T cells in the bone marrow, concurrent with an early and persistent block in B cell maturation (Pui J C, Allman D, Xu L, DeRocco S, Karnell F G, Bakkour S, Lee J Y, Kadesch T, Hardy R R, Aster J C, Pear W S (1999): Notch1 expression in early lymphopoiesis influences B versus T lineage determination. Immunity, 11, 299-308). These findings suggest that Notch1 plays an obligatory and selective role not only in T cell lineage induction but also in several cell fate decisions in T cell maturation.

Molecular Structure of Notch Protein Family Members

Notch proteins are 300 kD single-pass transmembrane receptors. The large extracellular domain contains 34-36 tandem EGF-like repeats and three cystein-rich Notch/Lin-12 (NL) repeats. Six tandem ankyrin or CdclO repeats, a glutamin-rich domain (opa), and a PEST sequence are found within the intracellular domain (Wharton K A, Johansen K M, Xu T, Artavanis-Tsakonas S (1985): Nucleotide sequence from the neurogenic locus notch implies a gene product that shares homology with proteins containing EGF-like repeats. Cell, 43, 567581). The intracellular domain is divided in the subdomains ICN1 and ICN2 with ICN1 containing the Cdc10 repeats (Pear, W. S., Aster, J. C., Hasserjian, R. B., Soffer, B., Sklar and Baltimore, D. (1996): Exclusive development of T cell neoplasms in mice transplanted with bone marrow expressing activated Notch alleles. Journal of Experimental Medicine, 183, 2283-2291).

Notch Ligands and Signaling Pathways

Genetic and molecular interaction studies resulted in the identification of a number of proteins that participate in transmitting and regulating Notch signal. In Drosophila, the two single -pass transmembrane proteins, Delta and Serrate (Delta and Jagged in vertebrates), have been identified as partially redundant extracellular Notch ligands (Artavanis-Tsakonas, S. Rand, M. D. and Lake, R. J. (1999): Notch signaling: cell fate control and signal integration in development. Science, 284, 770-776). The extracellular domain of these ligands, expressed on the surface of one cell, are thought to interact with the extracellular domain of the Notch protein expressed on a adjacent cell, resulting in the activation of the intra cellular notch domain. As a result of activation the intracellular domain is cleaved by Presenilin, a multiple transmembrane protein, (Struhl and Greenwald, 1999) and bind to different transcription factors such as Suppressor of Hairless (Su(H)) in Drosophila (CBF1/RJBK in mammals). Upon binding the Notch/Su(H) complex is translocated to the nucleus and upregulate the expression of the genes of the Enhancer of split Locus, which encode nuclear basic helix-loop-helix (bHLH) proteins (Bailey, A. M. and Posakony, J. W. (1995): Suppressor of hairless directly activates transcription of Enhancer of split complex genes in response to notch receptor activity. Genes & Development, 9, 2609-2622). The bHLH proteins, in turn, affect the regulation of downstream target genes, for instance, the genes of the Achaete-Scute complex in Drosophila, which contains proneuronal genes that encode proteins involved in the segregation of neuronal and epidermal lineages.

Beside of CBF1 and RJBK several other proteins reported to interact with the intracellular notch domain are e.g. Bcl3, a member of the IKB family (Jehn B M, Bielke W, Pear W S, Osborne B A (1999): Protective effects of notch-1 on TCR-induced apoptosis. J Immunol, 162, 635-638), Nur77, a protein involved in lymphoid development, Deltex, a cytoplasmic protein with putative SRC homology binding domains and Notchless, a WD 40-repeat containing protein (Royet, J., Bouwmeester, T. and Cohen, S. M. (1998): Notchless encodes a novel WD40-repeat-containing protein that modulates Notch signaling activity. The EMBO Journal, 17, 7351-7360).

Notchless Modulates Notch Signaling Activity

Several proteins have been identified as modifiers of the activity of Notch-family receptors. Deltex binds to the CDC10 repeats and positively regulates notch activity (Matsuno K, Eastman D, Mitsiades T, Quinn A M, Carcanciu M L, Ordentlich P, Kadesch T, Artavanis-Tsakonas S (1995) Human deltex is a conserved regulator of Notch signalling. Nature Genetics, 19, 74-78), Numb, Disheveled and Sel-10 binding reduce Notch activity. A novel protein, which reduces Notch activity by binding the intracellular ICN2 domain, is Notchless a novel WD40 repeat containing protein (Royet, J., Bouwmeester, T. and Cohen, S. M. (1998): Notchless encodes a novel WD40-repeat-containing protein that modulates Notch signaling activity. The EMBO Journal, 17, 7351-7360). Notchless (Nle) was first identified in a genetic screen for modifiers of Drosophila notch activity. Removing one copy of Notchless in Deltex mutant flies restores the deltex mutant wing to normal. This result suggests that Deltex and Notchless act in opposite directions as modifiers of notch activity in Drosophila wing development. Binding of Nle to the cytoplasmatic domain of Notch was confirmed by GST pull-down and immunoprecipitation assays. Experiments using the yeast two-hybrid system that Nle binds to the ICN2 domain of notch, but not to ICN1. This suggests that Nle is likely to oppose Deltex function indirectly through an opposing activity on Notch, and not by direct competition for binding.

The function of Notchiess appears to be to reduce Notch activity. Reduction or removal of Nle-expression increase Notch activity, but overexpression of Nle also leads to increased Notch activity in Drosophila and Xenopus. It is thought that NIe functions as a modulator to keep Notch activity levels in balance. Nle mutants show increases Notch activity but are viable even as homozygotes, indicating that the level of overactivation is not so severe as to be lethal. In this regard Nle functions like Deltex, which modulates the level of Notch activity, but which is not absolutely required for Notch to function.

Using degenerated PCR Primer directed against the N-terminal domain of mouse and human ESTs a Xenopus cDNA was isolated. Both predicted Notchless proteins has novel highly conserved N-terminal domains followed by nine WD40 repeats. The WD40 repeat is found in a wide variety of proteins of diverse function and is thought to be a protein interaction domain (Neer E J, Schmidt C J, Nambudripad R, Smith T F (1994): The ancient regulatory-protein family of WD-repeat proteins. Nature, 371, 297-300). Typically WD40 proteins contain seven repeats. Structure analysis of β-Transducin suggests that these form a propeller-like structure and that seven repeats can pack to make a flat cylinder (Neer E J and Smith T F (1996) G protein heterodimers: new structures propel new questions. Cell, 84, 175-178). Notchless is unusual in that it appears to contain nine WD40 repeats.

### REFERENCES

[1] G. R. Crabtree, Contingent genetic regulatory events in T lymphocyte activation, Science 243 (1989) 355-361.

f[2] C. H. June, Signal transduction in T cells, Curr. Opin. Immunol. 3 (1991) 287-293.

[3] R. H. Schwartz, Costimulation of T lymphocytes: the role of CD28, CTLA-4, and B7/BB1 in Interleukin-2 production and immunotherapy, Cell 71 (1992) 1065-1068.

[4] J. Banchereau, F. Bazan, D. Blanchard, F. Briere, J. Galizzi, C. van Kooten, Y. Liu, F. Rousset, S. Seeland, The CD40 antigen and its ligand, Annu. Rev. Immunol. 12 (1994) 881-922.

[5] D. J. Lenschow, T. Walunas, J. Bluestone, CD28/B7 system of T cell costimulation, Annu. Rev. Immunol. 14 (1996) 233-258.

[6] P. Linsley, J. Ledbetter, The role of the CD28 receptor during T cell responses to antigen, Annu. Rev. Immunol. 11 (1993) 191-212.

[7] A. Kupfer, S. L. Swain, S. J. Singer, The specific direct interaction of helper T cells and antigen-presenting B cells. II. Reorientation of the microtubule organizing center and reorganization of the membrane-associated cytoskeleton inside the bound helper T cells, J. Exp. Med. 165 (1987) 1565-1580.

[8] M. V. Parsey, G. K. Lewis, Actin polymerization and pseudopod reorganization accompany anti-CD3-induced growth arrest in Jurkat T cells, J. Immunol. 151 (1993) 1881-1893.

[9] N. Selliah, W. H. Brooks, T. L. Roszman, Proteolytic cleavage of -actinin by calpain in T cells stimulated with anti-CD3 monoclonal antibody, J. Immunol. 156 (1996) 3215-3221.

[10] R. Kojima, J. Randall, B. M. Brenner, S. R. Gullans, Osmotic stress protein 94 (Osp94): A new member of the Hspl O/SSE gene subfamily, J. Biol. Chem. 271 (1996) 12327-12332.

[11] J. Sambrook, E. F. Fritsch, T. Maniatis, Molecular Cloning: A Laboratory Manual, Cold Spring Harbour, New York, 1989.

[12] A. P. Feinberg, B. Vogelstein, A technique for radiolabeling DNA restriction endonuclease fragments to high specific activity, Anal. Biochem. 132 (1983) 6-13.

[13] F. Sanger, S. Nicklen, A. R. Coulson, DNA sequencing with chain-terminating inhibitors, Proc. Natl. Acad. Sci. USA 74 (1977) 5463-5467.

[14] M. Ashburner, P. Thompson, J. Roote, P. F. Lasko, Y. Grau, M. El Messal, S. Roth, P. Simpson, The genetics of a small autosomal region of Drosophila melanogaster containing the structural gene for alcohol dehydrogenase. Characterization of the region around the snail and cactus loci, Genetics 126 (1990) 679-694.

[15] S. Roth, F. S. Neuman-Silberberg, G. Barcelo, T. Schüpbach, Cornichon and the EGF receptor signaling process are necessary for both anterior-posterior and dorsal-ventral pattern formation in Drosophila, Cell 81 (1995) 967-978.

[16] R. Lehmann, Establishment of embryonic Drosophila oogenesis, Dev. Biol. 6 (1995) 25-38.

[17] F. S. Neuman-Silberberg, T. Schüpbach, The Drosophila dorsoventral patterning gene gurken produces a dorsally localized RNA and encodes a TGF-like protein, Cell 75 (1993) 165-174.

[18] J. V. Price, R. J. Clifford, T. Schüpbach, The maternal ventralizing locus torpedo is allelic to faint little ball, an embryogenic lethal, and encodes the Drosophila EGF receptor homolog, Cell 56 (1998) 1085-1092.

[19] J. Schlessinger, B. Geiger, Epidermal growth factor induces redistribution of actin and alpha-actinin in human epidermal carcinoma cells, Exp. Cell. Res. 134 (1981) 273-279.

[20] J. Singer, Intercellular communication and cell-cell adhesion, Science 255 (1992) 1671-1677.

[21] R. Pardi, L. Inverardi, C. Rugarli, J. R. Bender, Antigen-receptor complex stimulation triggers protein kinase C-dependent CD11a/CD18-cytoskeleton association in T lymphocytes, J. Cell. Biol. 116 (1992) 1211-1220.

[22] Pearson (1990). Methods in Enzymology, 183, pp. 63-98.

What is claimed is:

1. A polynucleotide encoding a TZap protein or a biologically active fragment thereof comprising a DNA sequence selected from the group consisting of
(i) DNA sequences comprising a nucleotide sequence encoding the amino acid sequence depicted in SEQ ID NO.2, 4 or 6;
(ii) DNA sequences comprising the nucleotide sequence depicted in SEQ ID NO. 1, 3 or 5;
(iii) DNA sequences comprising a nucleotide sequence encoding a fragment or derivative of the protein encoded by the DNA sequence of (i) or (ii);
(iv) DNA sequences the complementary strand of which hybridizes with and which is at least 70% identical to the polynucleotide as defined in any one of (i) to (iii);
(v) DNA sequences the nucleotide of which is degenerate to the nucleotide sequence of a DNA sequence of any one of (i) to (iv); and
(vi) DNA sequences comprising a nucleotide sequence encoding a TZap-consensus sequence of the amino acid sequences depicted in SEQ ID NOs: 2, 4 and 6.

2. A nucleic acid molecule of at least 15 nucleotides in length hybridizing specifically with a polynucleotide of claim 1 or with a complementary strand thereof.

3. A vector comprising the polynucleotide of claim 1 or the nucleic acid molecule of claim 2.

4. The vector of claim 3, wherein said polynucleotide or nucleic acid molecule is operably linked to regulatory sequences allowing for the transcription and optionally expression of said nucleic acid molecule.

5. A host cell comprising a polynucleotide of claim 1 or the vector of claim 3 or 4.

6. A method for the production of a TZap protein or a biologically active fragment thereof comprising:
(a) culturing the host of claim 5 under conditions allowing for the expression of the protein; or
(b) in vitro translation of the polynucleotide of claim 1;
and recovering the protein produced in (a) or (b).

7. A TZap protein or biologically active fragment thereof encoded by the nucleic molecule of claim 1 or produced by the method of claim 6.

8. An antibody specifically recognizing the protein of claim 7.

9. A normal cell that has been modified to express the protein of claim 7 or the antibody of claim 8.

10. A pharmaceutical composition comprising the polynucleotide of claim 1, the nucleic acid molecule of claim 2, a vector of claim 3 or 4, the cell of claim 5, the protein of claim 7, the antibody of claim 8 or an antisense construct capable of inhibiting the expression of a polynucleotide of claim 1, and optionally a pharmaceutically acceptable carrier.

11. The pharmaceutical composition of claim 10 for use in cell or organ transplantation, for the treatment of autoimmune, allergic or infectious diseases, or for the treatment of tumors.

12. A diagnostic composition comprising a polynucleotide of claim 1, the nucleic acid molecule of claim 2, the vector of claim 3 or 4, the cell of claim 5 or 9, the protein of claim 7, or the antibody of claim 8; and optionally, at least one component which is labeled.

13. A method of diagnosing a pathological condition or a susceptibility to a pathological condition in a subject related to a disorder in the immune system comprising:
(a) determining the presence or absence of a mutation in the polynucleotide of claim 1; and
(b) diagnosing a pathological condition or a susceptibility to a pathological condition based on the presence or absence of said mutation.

14. A method of diagnosing a pathological condition or a susceptibility to a pathological condition in a subject related to a disorder in the immune system comprising:
(a) determining the presence or amount of expression of the protein of claim 7 in a biological sample; and
(b) diagnosing a pathological condition or a susceptibility to a pathological condition based on the presence or amount of expression of the protein.

15. A method for identifying a binding partner to a TZap protein comprising:
(a) contacting a protein of claim 7 with a compound to be screened; and
(b) determining whether the compound effects an activity of the protein.

16. A method for identifying leukocyte activating or co-stimulating compounds or for identifying inhibitors of leukocyte activation and stimulation comprising
(a) culturing lymphocytes or monocytes in the presence of the protein of claim 7, the antibody of claim 8, the cell of claim 5 or 9 and, optionally, in the presence of a component capable of providing a detectable signal in response to leukocyte proliferation, with a compound to be screened under conditions permitting interaction of the compound with the (poly)peptide, antibody or cell(s); and
(b) detecting the presence or absence of a signal generated from the interaction of the compound with the cells.

17. A method of identifying modulators of immune responses comprising the steps of:
(a) incubating a candidate compound with TZap7/A, TZap7/B and/or TZap7;
(b) assaying a biological activity as described above; and
(c) determining if a biological activity of TZap has been altered.

18. A method for the production of a pharmaceutical compositions comprising the steps of the method of any one of claims 15 to 17 and formulating and optionally synthesizing the compound identified in step (b) in a pharmaceutically acceptable form.

19. A method for determining the status of an immune response comprising analyzing the presence of the polynucleotide of claim 1 or the protein of claim 7.

20. Use of the polynucleotide of claim 1, the nucleic acid molecule of claim 2, the vector of claim 3 or 4, the protein of claim 7, the antibody of claim 8, the cell of claim 5 or 9 or the compound identified according to the method of any on of claims 13 to 16 for the preparation of a composition for diagnosing or the treatment of acute and chronic diseases, involving T cell activation and Th1 and Th2 immune response, for the treatment of acute and chronic rejection of allo-and xeno organ transplants and bone marrow transplantation, for the treatment of rheumatoid arthritis, lupus erythramatodes, multiple sclerosis, encephalitis, vasculitis, diabetes mellitus, pancreatitis, gastritis, thyroiditis, for the treatment of maligne disorders of T, B or NK cells, for the treatment of asthma, lepramatosis, Helicobacter pylori associated gastritis or for the treatment of skin tumors, adrenal tumors or lung tumors, wound healing, growth disorders, inflammatory and/or infectious diseases.

21. Use of a polynucleotide of claim 1, a nucleic acid molecule of claim 2 or the antibody of claim 8 for the detection of leukocyte activation.

22. Use of claim 20, wherein said leukocyte is a B cell, T cell, NK cell and/or monocyte.
